SEQUENCE LISTING (34 sequences)

SEQ ID NO: 1 - DNA, 2191 bp, synthetic construct
  source 1..2191: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaaggaggaggaagcatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgct

SEQ ID NO: 2 - DNA, 2211 bp, synthetic construct
  source 1..2211: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaaggaggaggaagcatgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgta

SEQ ID NO: 3 - DNA, 2200 bp, synthetic construct
  source 1..2200: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaaggaagcggaggaggaggaagcatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgct

SEQ ID NO: 4 - DNA, 2220 bp, synthetic construct
  source 1..2220: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaaggaagcggaggaggaggaagcatgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgta

SEQ ID NO: 5 - DNA, 2218 bp, synthetic construct
  source 1..2218: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaaggaagcggagaagcagcagcaaaagaagcagcagcaaaaatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgct

SEQ ID NO: 6 - DNA, 2238 bp, synthetic construct
  source 1..2238: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaaggaagcggagaagcagcagcaaaagaagcagcagcaaaaatgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgta

SEQ ID NO: 7 - DNA, 2509 bp, synthetic construct
  source 1..2509: mol_type = other DNA
atgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgttaggaggaggaagcatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgct

SEQ ID NO: 8 - DNA, 2529 bp, synthetic construct
  source 1..2529: mol_type = other DNA
atgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgttaggaggaggaagcatgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgta

SEQ ID NO: 9 - DNA, 2518 bp, synthetic construct
  source 1..2518: mol_type = other DNA
atgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgttaggaagcggaggaggaggaagcatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgct

SEQ ID NO: 10 - DNA, 2538 bp, synthetic construct
  source 1..2538: mol_type = other DNA
atgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgttaggaagcggaggaggaggaagcatgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgta

SEQ ID NO: 11 - DNA, 2536 bp, synthetic construct
  source 1..2536: mol_type = other DNA
atgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgttaggaagcggagaagcagcagcaaaagaagcagcagcaaaaatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgct

SEQ ID NO: 12 - DNA, 2556 bp, synthetic construct
  source 1..2556: mol_type = other DNA
atgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgttaggaagcggagaagcagcagcaaaagaagcagcagcaaaaatgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgta

SEQ ID NO: 13 - DNA, 2191 bp, synthetic construct
  source 1..2191: mol_type = other DNA
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctggaggaggaagcatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaa

SEQ ID NO: 14 - DNA, 2509 bp, synthetic construct
  source 1..2509: mol_type = other DNA
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctggaggaggaagcatgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgtta

SEQ ID NO: 15 - DNA, 2200 bp, synthetic construct
  source 1..2200: mol_type = other DNA
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctggaagcggaggaggaggaagcatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaa

SEQ ID NO: 16 - DNA, 2518 bp, synthetic construct
  source 1..2518: mol_type = other DNA
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctggaagcggaggaggaggaagcatgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgtta

SEQ ID NO: 17 - DNA, 2218 bp, synthetic construct
  source 1..2218: mol_type = other DNA
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctggaagcggagaagcagcagcaaaagaagcagcagcaaaaatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaa

SEQ ID NO: 18 - DNA, 2536 bp, synthetic construct
  source 1..2536: mol_type = other DNA
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctggaagcggagaagcagcagcaaaagaagcagcagcaaaaatgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgtta

SEQ ID NO: 19 - DNA, 2211 bp, synthetic construct
  source 1..2211: mol_type = other DNA
atgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgtaggaggaggaagcatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaa

SEQ ID NO: 20 - DNA, 2529 bp, synthetic construct
  source 1..2529: mol_type = other DNA
atgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgtaggaggaggaagcatgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgtta

SEQ ID NO: 21 - DNA, 2220 bp, synthetic construct
  source 1..2220: mol_type = other DNA
atgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgtaggaagcggaggaggaggaagcatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaa

SEQ ID NO: 22 - DNA, 2538 bp, synthetic construct
  source 1..2538: mol_type = other DNA
atgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgtaggaagcggaggaggaggaagcatgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgtta

SEQ ID NO: 23 - DNA, 2238 bp, synthetic construct
  source 1..2238: mol_type = other DNA
atgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgtaggaagcggagaagcagcagcaaaagaagcagcagcaaaaatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaa

SEQ ID NO: 24 - DNA, 2556 bp, synthetic construct
  source 1..2556: mol_type = other DNA
atgaactcacgtcgttcagctaattatcaaccaaacctctggaattttgagtttctgcagagtttggaaaacgaccttaaggttgagaaacttgaggagaaggcgacgaaactggaagaggaagttcgctgcatgatcaatcgtgtggatacacaacctctgagtttgcttgaactgattgatgacgtgcagagactcggactgacgtacaagttcgaaaaagatatcatcaaagctctggagaatatcgtccttctggatgagaacaagaaaaataagtcagacttgcacgcgacagcgctgagctttcgtttattaagacaacacggctttgaagtgtcacaagacgtttttgagcggttcaaggataaggaggggggtttcagtggggaactcaagggtgacgtgcaaggactcctcagtctgtacgaggcgagttatctcgggttcgagggtgagaatttattagaagaggcacggactttttctataacacacttaaaaaataacttgaaagaaggtataaacactaaagtcgccgaacaggtttcacatgctctggagctcccttatcaccagagactccaccgtctggaggctcgttggttcctggataaatatgaaccgaaggagccacatcatcaacttctgcttgaattggctaagttagactttaacatggttcagacactgcaccagaaggaactgcaggacctgtcacgctggtggactgagatgggtttagcttccaagcttgacttcgttcgtgacagactcatggaggtttatttttgggccttgggaatggcgcctgatccgcaatttggtgagtgtcggaaagcggttacgaaaatgttcgggctggtgaccattatcgacgacgtgtatgatgtttatgggacactggatgaacttcaactctttacggatgcagtcgaacgctgggatgtgaatgcgataaacaccctccctgattacatgaaattgtgctttttagcgctctataatactgttaacgatacatcctactcaatcctcaaagaaaaaggacacaataaccttagttatcttactaaatcttggcgcgagctgtgcaaggccttcctgcaggaagctaaatggagtaataacaagataattcctgcatttagcaagtacttggaaaatgcaagtgtaagttcttccggtgtagctttgctcgccccaagctacttctcagtatgtcaacagcaggaggatattagtgaccatgcgcttcgcagtttgacggacttccacggactcgtccgctcttcatgcgtaattttccgtttgtgcaacgatctggccaccagcgcggccgagttggagcgtggagaaacgactaatagcatcataagctatatgcatgaaaatgacggaacatccgaagagcaagcacgtgaagagctccggaagcttatagacgcagaatggaagaagatgaaccgggagcgcgtatcagatagtaccctcttaccaaaggcttttatggaaattgcggtgaatatggcgcgcgtatcccattgtacttaccaatatggggacggcttgggacgtcctgattatgcgacagagaatcggattaaattattgttaatcgacccattcccaatcaatcaactgatgtacgtaggaagcggagaagcagcagcaaaagaagcagcagcaaaaatgaccgccgataataactctatgcctcacggggctgtatccagctacgcgaaattagtacaaaatcagaccccggaagacatcctggaggaattcccagaaatcatccctttacagcaacgtcctaacacacgtagtagtgagacatctaacgatgaaagcggggaaacctgcttttctgggcatgacgaggagcagattaagcttatgaacgagaactgcattgttttggattgggacgataatgctatcggagcgggtactaaaaaggtttgccatttaatggagaatattgaaaaaggactgctgcatcgcgcgttctccgtattcatcttcaatgagcagggtgaacttcttttgcaacaacgcgccaccgaaaagatcacgttcccagatctttggaccaacacgtgttgctcacatcctttgtgtatcgacgatgaactgggattaaagggtaagttggacgacaagattaagggagccatcacggctgccgtgcgtaagcttgaccatgagttgggaattcccgaagatgaaaccaaaacgcgtggcaagttccatttcctgaatcgtatccattacatggcaccttccaatgaaccatggggtgagcatgaaatcgactatattttattttacaagatcaatgcgaaagagaaccttaccgtcaacccgaatgtgaatgaggtccgcgatttcaaatgggttagcccaaacgacctgaaaacgatgtttgccgatcccagctacaagtttacaccgtggtttaagattatctgcgagaattacttgttcaattggtgggagcagcttgacgacttatcggaagtagagaatgaccgtcagatccatcgcatgtta

SEQ ID NO: 25 - protein, 544 aa, unidentified
MEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFCFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLSGSLFAKPFVETAINLARQSHCTYHNGDAHTSPDELTRKRVLSVITEPILPFER

SEQ ID NO: 26 - protein, 551 aa, unidentified
MNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 27 - protein, 4 aa, unidentified
GGGS

SEQ ID NO: 28 - protein, 7 aa, unidentified
GSGGGGS

SEQ ID NO: 29 - protein, 13 aa, unidentified
GSGEAAAKEAAAK

SEQ ID NO: 30 - protein, 182 aa, unidentified
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 31 - protein, 288 aa, unidentified
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 32 - DNA, 9909 bp, synthetic construct
  source 1..9909: mol_type = other DNA
aggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcactagtaacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatccctaggtataaacgcagaaaggcccacccgaaggtgagccagtgtgactctagtagagagcgttcaccgacaaacaacagataaaacgaaaggcccagtctttcgactgagcctttcgttttatttgatgcctggagatccttactcgagtttggatcctaatcacttatccagatacgtttccgggtccttctccttgcgaacaccccagtcggcctgcgtaacatccagccattggactttgctgaaacgtttgtcgttggccgtttgtgcgcgcagatcgacgtcctgtttcgtgatgacggcaatggcgtcgtaaccacccgcgcccggaatcaggcacgttaacacgccctttaaggtttgacagtcgtccagcagagacgtctggaccggtggttcgatatccgcgccgctctccttcgtaattttgcggaagctacggcgaatcgttgccacggcatcgcggacctccgtgatctccgggtacttctggcaggtgcagtcattgcgctccaggctctcgaagatttggtcgctgtagtcgtcgtgggtttcgtgcaggcggtccagcttgctcagaccatccatgaaacggctattggcgtggtccagctccgtgtaaatcttcaggctttccggcatgtggctgtcgtaccagtttttcactttctgcaccagcttaaccgtttcgctgccgttcttgatatcgcccatccataacgttaagccgctcggcaggtgattagacttaatggtgatgttccagtcttcttcgtccaccagatgcgccagtttgctaccatacgtcgcagagccgatgtccggcagattgctgatcagggccggcggaaagcggcgatagcggatagagccataggcggccgctgccacatcaaaaccagagccaatcttaccttgggcctgacaatgtgccacctgcgccaggttatgaatcacttcgcggtacttgtccacgttgttctccaggtcgctcacgaagaagctcgctaacgccgtcgttaagacggtaactaaaccggcgctagaacccagacccgtcttcggcacctcctcaatgcgatggctgtggaagctcaggcggcggttaccacgatgctccgtaacagagtcctcttggctgtggtacgcgtcgtcgctgaaaatgtcgatgacgaacaggttgcggttgcagtaatcgtccatattcggcttgaagtagctgaagacgttggcaatgaccttctcgataaatgggttcttgctgccaccaatgctaaccgggatgaagccgctctttgggctaatgtggtacagccactcgccgtccttgaactgcttgctcttcacacgcacttcgaacttgtcagagccctgcaggctaccatacgggtgggcaactgcatgcatacgggcagataagccgacgacgaacgcctcgtacttggtgtctaacaccaggtagccaccggccagcagtgccttacctggcgcgctgaatgcacgtaattcgctcatatgatctccctcctagatccttaagacgtccacggcagattggtgttacccggcagtaacaggtcgtcgatctgctgcttcgtggtcgtcttgttttcgaacagctggaacaccaggcttttgatcttcaggtccttgttcagatttttggcgctcagcaggcaacagcccgtgccgcccaggtccgtctcgaacgtctcgtagctgaagtcgtcctgcagcttcttcttgaagctgtcgatttgctcctgcgtaatgtcacggcgcagcagcgttaaagagcagccaccgccacctgcacccgttaatttcgtagagccaatgcgcaggtcgtcgctcaggtttttgatcagttccaggcccgggtgagacacaccgatagaaaccagcaggccgtgattgatacggatcagctccagcagctgctcgtacagctcattgttggtttccaccgcctcatcgtcggtgcccttgcacttgctcagcttggtcatgatctccagaccctgcagcgcgcactcgcccatcgcgtccaggatcggcttcatcacctccgggaacttttcggtgactaaaacacgcacgcgggcgactaaatccttggtgctgcgtgggatacgggtgtaggtcagaatcatcgggatcgccgggaagtcgtccaggaacttgaagttgttcgtgttgatcgtgccgttgtggctgtctttctcgaacagcagggcgttgccatacgtggcaactgcgttgtcaatgccgctcggggtgccatgaatgcacttctcgccgataaaggcccactggttcacgatgtgcttgtcgttttcgctcagtttctccaggtcgttgctgccaatcaggccacccagataggccatcgccagcgccaggctcacgctgatagatgcgctagagcctaagcctgcgccaatcggcagcgtgctcttcaggctgaacttgatgttctttgcgtgcgggcacaggcaaacgaacatgtacaggaagcagaacgcggcgtggtaatggaagctctcgcttaactgcgctaacagcgggtccagcagagacaccagctcctgagacagaccgtcggttgcttgctgggctttggccagcttttggctgttcacctggtcctccgtgatcgcgttgaagtcgttgatgctccacttgtggttgaagctgatgtccggaaagtccagctcgatcgtgtccggtgcagaagattcgctgatcagcaggtaggtgcgtaacgcagacacagaggcggcgacggccggcttattgtacaccgcagagtgctcgccgaagatgatcaccttacctggcgcagacgtcaggaatggcagagacatggttatttcctcctagatcctttagatccagatcctaattattgttgtctaatttcttgtaaaatatgttccgctgcttgtgtatttgcacggggttcttttttcaaagcttcagctactttagcaatttcatcaccttttgcccctacaacgatagctaatgatttatattgtaaactcatatgaccttgttgaataccttctgacacaagcgcgcgacatgctgcaaagttttgcgctaaaccaacggcagcaactacatgacctaattcttgtgctgactctacatttagtagctctaatgaagctttagcaattggtaatacttttgtaccaccgccaacgattgccaatgtcataggcacttcaattgtaccaatcaatcgttgacgatcttgatcgtaacgccatgtagcaataccacgatactgtccgtcacgactcgcgtatgcatgcgcacttgcttctgcaccacgcgtatcatttcctgttgctaaaacaacagcatgtatgccattcataacacctttattatgtgttgctgcacgatgaatatctacttgggccaatacagaagcacgttccattcgtttggcaacctcttctccagttctctcgccccttgctaaatctttaacatcaatttcgccttgaactttaacaacggacgctgttgcatgattggataaaatactcattaaaatgtcgctttgcggaaattcattttttaaaaatgcagttatggcctctaaaatcgtattaagcatattagcgcccatagcatctttcgtatcaacaaatacttttaaagatagtaactgttgctcaggaaatgtatcaatcgctatacgttggtaaccaccaccacgcgctttaatagaaggatatgcctcatccgcaattttatgaatttgcttttctaaagctttaatgtctgctgataatttttcagtatcgtcaacgccatcaaagacgatttgacctatcataatacgttcagaagataccgttttaaatccgccagtctgattcactagctttgcaccataactagctgcagcgacaactgaaggctcttccaccatcataggtacaacatatgccttatcgtccacaatgatattcggtaataatccaacgggtaatgcaccttgcgcgatgacattttcaattaaactattggctacttcttcatcgattaatggatgattcagtaaaatgtcgaattgttcttctgataaccattgcttatcaaccaattgttgtaacttttctttacgagataaatgtcgaaaattcttatctaaactttgcatggacatggtctgtttcctgaatagatcctaattactctggtctgtgatattcgcgaacgttattttcaatatttgataagtagaaaatatgacgatcttcatgaacagcatcttgttcttcgtcaaattcaacgtcatcaaaacgtttgaagaatgtttcatatgcatcaacagatacttcagtacggttatttaataatgctttatgtgcagcttgatctaaatgatctttgtagccttcaactaatgtcgcactataaaattcaccaactgaacctgagccataactgaataaaccgattgtttcaccagcttgtaaatctcgattttcaagtaatgatattaggcttaaatataatgatccagtataaatattaccgacataacggttataatctacagcatcttcatatcctgaacgtaaacgctcttgagttgtttcatcagcgttatcaatgattgactctaatgcctttttacccatttttgtaaatggaacatggaagcatagagatgcgaagtcagctagcgacttaccttgacgttttgcgtattcattccagctttgttggaatgagcggatataagcatctttagataatgcaccatcaactaatggatatttatgtccagttggacgccagaaatcataaacgtcttcagtgtaagcaacagcatcttcatttaatgccaaaatgcttggattatgtgcaataaccatcgcaactgcgccagcaccttgtgttggctcgccgcctgagttcaatccataacgtgctgtatctgtagcaataactaatactttttcattcggtctagttgctaaataatctttagctaattgaattgctggtgttgcagcataacaagcttctttcatttcaaagcagcgtgcaaaaggttgaatacctaataagttgtgaatttgaacagcggctgctttagcagcatcaactgctgattcagttgccacaattaccataccaattttctttttgtcttcgtctgttataatgtccttagcagcgttagcgcccattgaaacgatgtcttggtttacaggactaacagccatttcagtttgaccaattccaattaaaaatttgtttgggtctacttggcgtgcttctgctaatttagccatgtctacatagtactttggaacgtaaaagtttattttatcgatacctattgtcataaggtttctcctgaatagatcctcaattcaaccgttcaatcaccatcgcaattccctgaccgccgccaatgcacagtgttgccagccccagcgttttatcgcgtgcctgcatggcatgtaatagtgtgaccagaatacgagcaccactggcaccgataggatgcccgagcgcgatggccccgccgttgacattcactttctcagaatcaaagcccaggtttttcccaacggcaaggaactgtgcagcaaatgcttcattagcctcaatgagatcaatatccgccagttgcagccccgccagttgtaacgctttttgcgtggcaggtactggccccatacccatcaatgcggggggcacgccaccgctggcataacttttaatgcgagccaggggggtaaggcctgctgccagcgccgcagattcttccataatcaccagagcggcagcaccgtcgttaataccagacgcgttcccagcggtgactgttcctgctttatcgaaggccgggcgcaatgcacctaacgcttcagccgttgagttcgctttcgggaactcgtcttgactgaagacgaaggttttctttcgagtgacaacatttaccgggacgatttcggctgtaaaagcaccggactcaattgcggctgccgctttacgctgtgaatgtagcgccagttcatcctgcatttcacgggtaattccgtactctttagccacgttttcggcggtaatccccatatgataaccatgggtggcgcacatcaggccatcgcgcaggattacgtcataaacctgtccgtctccaagacgataaccagagcgtgcttttgcatcgagtaagtagggggctaaactcatattttccataccccccgccacaatgctctgcgcctgacctgcctgaatggcctgggcggcaagcgccacacttttaagacccgaaccacatactttattgaccgtgaatccgcacaccgtttctgccagcccgctttttaacagtgcctgacgcgccggattttgccccagcccggcttgtaacacgttacccataatcacttcatcaacgtgttgtgaatcgatttttgcacgttcaatggcggctttaattactgtcgcccccaggtcgatggcgctggtggaagcgagtgaaccgttaaaactaccgatagcagtacgtaccgcactgacgatgacacaatttttcattttatattcctcctaagatcttttgaattctgaaattgttatccgctcacaattccacacattatacgagccggaagcataaagtgtaaagcctggggtgcctaaacgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgacgtcgatatctggcgaaaatgagacgttgatcggcacgtaag

SEQ ID NO: 33 - DNA, 7617 bp, synthetic construct
  source 1..7617: mol_type = other DNA
gctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccaggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgggaattcaaaagatcttttaagaaggagatatacatatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataacagaatttaaaactaatacgcgggaggttttttatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaaggatccaaactcgagttcggatctaggaggattatgagatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataactgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtg

SEQ ID NO: 34 - DNA, 7528 bp, synthetic construct
  source 1..7528: mol_type = other DNA
aacagaatttgcctggcggcagtagcgcggtggtcccacctgaccccatgccgaactcagaagtgaaacgccgtagcgccgatggtagtgtggggtctgcccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtgtccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtgtcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgggaattcaaaagatcttttaagaaggagatatacatatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataacagaatttaaaactaatacgcgggaggttttttatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaaggatccaaactcgagttcggatctaggaggattatgagatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtatctgcaattgccaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

(19) United States
(12) Patent Application Publication
(10) Pub. No.: US 2026/0117263 A1
(43) Pub. Date: Apr. 30, 2026

(54) GENETICALLY ENGINEERED PEPTIDE SUPERSTRUCTURE AND MICROBE FOR PRODUCTION OF ISOPRENE AND METHOD OF PRODUCTION THEREOF

(71) Applicant: CleanJoule, Inc., Salt Lake City, UT (US)

(72) Inventors: Jonathan Cloyd Greenhalgh, Salt Lake City, UT (US); Robert Andrew Price, Salt Lake City, UT (US)

(73) Assignee: CleanJoule, Inc., Salt Lake City, UT (US)

(21) Appl. No.: 19/374,049

(22) Filed: Oct. 30, 2025

Related U.S. Application Data
(60) Provisional application No. 63/713,890, filed on Oct. 30, 2024.

(51) Int. Cl. C12P 5/00; C12N 9/88; C12N 9/90

(52) U.S. Cl. CPC C12P 5/007; C12N 9/88; C12N 9/90; C12Y 402/03027; C12Y 503/03002

(57) ABSTRACT

A genetically engineered microbe capable of producing isoprene, wherein the genetically engineered microbe including a heterologous nucleic acid construct encoding at least a portion of a peptide superstructure; the peptide superstructure including a first portion including a first binding site configured to catalyze a first chemical reaction, wherein the first reaction converts a substrate to an intermediate; and a second portion including a second binding site configured to catalyze a second chemical reaction, wherein the second binding site is located in proximity to the first binding site and the second chemical reaction converts the intermediate to isoprene.

### REFERENCE TO RELATED APPLICATIONS

This application claims the benefit of priority of U.S. Provisional Application Ser. No. 63/713,890, filed on Oct. 30, 2024, and entitled “GENETICALLY ENGINEERED PEPTIDE SUPERSTRUCTURE AND MICROBE FOR PRODUCTION OF ISOPRENE AND METHOD OF PRODUCTION THEREOF,” which is incorporated by reference herein in its entirety.

### FIELD OF THE INVENTION

The present invention generally relates to the field of metabolic engineering. In particular, the present invention is directed to a genetically engineered peptide superstructure and microbe for production of isoprene.

### REFERENCE TO SEQUENCE LISTING

This specification includes a sequence listing submitted herewith, which includes the file entitled 1656-002USU1.xml having the following size: 113,663 bytes which was created Oct. 20, 2025, the contents of which are incorporated by reference herein.

### BACKGROUND

Isoprene is an important commodity chemical used in a wide range of industrial applications, from the production of synthetic rubber for tires and coatings to uses in adhesives and development of specialty elastomers. Current production of isoprene is derived mostly from unsustainable petrochemical sources and often involves resource-intensive methods that require extensive land use, high temperature, and/or use of precious metals. Biosynthesis of isoprene, while showing some progress, is often plagued by competing side reactions that limit the yield of isoprene.

### SUMMARY OF THE DISCLOSURE

In an aspect, a genetically engineered microbe capable of producing isoprene, wherein the genetically engineered microbe including a heterologous nucleic acid construct encoding at least a portion of a peptide superstructure; the peptide superstructure including a first portion including a first binding site configured to catalyze a first chemical reaction, wherein the first reaction converts a substrate to an intermediate; and a second portion including a second binding site configured to catalyze a second chemical reaction, wherein the second binding site is located in proximity to the first binding site and the second chemical reaction converts the intermediate to isoprene.

In another aspect, a genetically engineered peptide superstructure for production of isoprene, the superstructure including a first portion including at least a functional fragment of isoprene synthase (IspS) configured to catalyze a first chemical reaction, and a second portion including at least a functional fragment of isopentenyl diphosphate (IPP) isomerase configured to catalyze a second chemical reaction, wherein the second chemical reaction converts a substrate to an intermediate, wherein the second portion is covalently linked to the first portion through a linker, and wherein the intermediate is converted into isoprene by the first chemical reaction.

In another aspect, a genetically engineered peptide superstructure for production of isoprene, the superstructure including a first portion including at least a functional fragment of isopentenyl diphosphate (IPP) isomerase configured to catalyze a first chemical reaction, wherein the first chemical reaction converts a substrate to an intermediate, and a second portion including at least a functional fragment of isoprene synthase (IspS) configured to catalyze a second chemical reaction, wherein the second portion is covalently linked to the first portion through a linker, and wherein the intermediate is converted into isoprene by the second chemical reaction.

In another aspect, a method of producing isoprene using a genetically engineered peptide superstructure, the method including culturing a genetically engineered microbe under suitable conditions, wherein the genetically engineered microbe includes a heterologous nucleic acid construct encoding at least a portion of a peptide superstructure, and the superstructure includes a first portion including a first binding site and a second portion including a second binding site, the second portion being covalently linked to the first portion through a linker; and providing a substrate to the cultured genetically engineered microbe, wherein the first binding site is configured to bind to the substrate and catalyze a first chemical reaction that converts the substrate to an intermediate, and the second binding site is configured to catalyze a second chemical reaction that converts the intermediate to isoprene.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic illustration of an exemplary embodiment of a genetically engineered peptide superstructure for production of isoprene and its working principle;

FIG. 2 is an exemplary scheme of the methylerythritol phosphate (MEP) pathway, the mevalonate (MVA) pathway, and additional metabolic pathways associated thereto that are relevant for biosynthesis of isoprene;

FIGS. 3A-B are additional exemplary schemes of expanded metabolic pathways that are relevant for biosynthesis of isoprene; potential gene targets are also included, wherein the gene targets may be overexpressed or knocked down/knocked out to improve the yield of isoprene;

FIG. 4 is a schematic illustration of an exemplary embodiment of a method for producing isoprene using a genetically engineered peptide superstructure; and

FIG. 5 is an exemplary experimental result of isoprene production for IDI and IspS fusion, media only, and empty vector (P1+P2 system without IspS) control;

FIG. 6 is an exemplary result of the fusion construct containing the IDI_Linker2_IspS sequence FIG. 7 is an exemplary result of the fusion construct containing the IDI_Linker2_IspS sequence;

FIG. 8 is an exemplary result of the fusion construct containing the IDI_Linker3_IspS sequence;

FIG. 9 is an exemplary result of the fusion construct containing the IspS_Linker1_IDI sequence;

FIG. 10 is an exemplary result of the fusion construct containing the IspS_Linker2_ID sequence;

FIG. 11 is exemplary result of the fusion construct containing the IspS_Linker3_ID sequence; and

FIGS. 12A-B are exemplary embodiments of plasmid vector maps used for P1+P2 systems.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to genetically engineered peptide superstructures for production of isoprene, genetically engineered microbes configured to encode such peptide superstructures, and methods of production related thereto.

Peptide superstructure includes a first portion and a second portion. In one or more embodiments, a second portion is covalently linked to the first portion through a linker. In some cases, linker may include a peptide linker.

The first portion includes a first binding site configured to catalyze a first chemical reaction that converts a substrate to an intermediate.

In one or more embodiments, the first portion may include at least a functional fragment of isopentenyl diphosphate (IPP) isomerase. In one or more embodiments, substrate may include IPP. In one or more embodiments, intermediate may include dimethylallyl pyrophosphate (DMAPP).

The second portion includes a second binding site configured to catalyze a second chemical reaction converting the intermediate to isoprene. The second binding site is located in proximity to the first binding site. In one or more embodiments, the second portion may include at least a functional fragment of isoprene synthase (IspS).

In one or more embodiments, peptide superstructure may be produced in a genetically engineered microbe.

In some cases, genetically engineered microbe may include bacteria or fungi. In some cases, genetically engineered microbes may include Escherichia including Escherichia Coli, Lactococcus including Lactococcus lactis, Vibrio including Vibrio natriegens, Saccharomyces including Saccharomyces cerevisiae, Aspergillus including Aspergillus niger, Aspergillus nidulans, and/or Aspergillus oryzae, Yarrowia including Yarrowia lipolytica, Bacillus including Bacillus subtilis, Clostridium including Clostridium ljungdahlii, and/or Pseudomonas including Pseudomona sputida, among others. In some cases, genetically engineered microbes may include a Crabtree-negative microbe. In some cases, genetically engineered microbes may include a diploid strain. In some cases, genetically engineered microbes may include balanced methylerythritol phosphate (MEP) and mevalonate (MVA) pathways.

Genetically engineered microbe includes a heterologous nucleic acid construct encoding at least a portion of peptide superstructure.

The method of producing isoprene using genetically engineered peptide superstructure includes culturing the genetically engineered microbe under suitable conditions and providing substrate to the cultured genetically engineered microbe to produce isoprene.

Aspects of the present disclosure may be used to provide a sustainable means of producing isoprene and similar value-added chemicals. Aspects of the present disclosure may be used to reduce the dependence on fossil fuels and mitigate the negative environmental impacts thereof. Exemplary embodiments illustrating aspects of the present disclosure are described below in the context of several specific examples.

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

It is understood that the acts described below are meant as a general overview and demonstration of an exemplary method, and that the method may include different and/or additional acts as described herein or otherwise.

While the present invention will be described as having particular configurations disclosed herein, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

It is to be understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (for example, a step is performed by or with the assistance of a human).

For the purposes of this disclosure, including the claims, the phrase “at least some” means “one or more” and includes the case of only one. Thus, for example, the phrase “at least some ABCs” means “one or more ABCs” and includes the case of only one ABC.

For the purposes of this disclosure, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

For the purposes of this disclosure, the term “portion” means some or all. Therefore, for example, “A portion of X” may include some of “X” or all of “X”. In the context of a conversation, the term “portion” means some or all of the conversation.

For the purposes of this disclosure, including the claims, the phrase “using” means “using at least” and is not exclusive. Thus, for example, the phrase “using X” means “using at least X”. Unless specifically stated by use of the word “only”, the phrase “using X” does not mean “using only X”.

For the purposes of this disclosure, including the claims, the phrase “based on” means “based in part on” or “based, at least in part, on” and is not exclusive. Thus, for example, the phrase “based on factor X” means “based in part on factor X” or “based, at least in part, on factor X”. Unless specifically stated by use of the word “only”, the phrase “based on X” does not mean “based only on X”.

In general, for the purposes of this disclosure, including the claims, unless the word “only” is specifically used in a phrase, it should not be read into that phrase.

For the purposes of this disclosure, including the claims, the phrase “distinct” means “at least partially distinct”. Unless specifically stated, distinct does not mean fully distinct. Thus, for example, the phrase “X is distinct from Y” means that “X is at least partially distinct from Y” and does not mean that “X is fully distinct from Y”. Thus, for the purposes of this disclosure, including the claims, the phrase “X is distinct from Y” means that X differs from Y in at least some way.

It should be appreciated that the words “first”, “second”, and so on, in the description and claims, are used to distinguish or identify, and not to show a serial or numerical limitation.

Similarly, letter labels (for example, “(A)”, “(B)”, “(C)”, and so on, or “(a)”, “(b)”, and so on) and/or numbers (for example, “(i)”, “(ii)”, and so on) are used to assist in readability and to help distinguish or identify, and are not intended to be otherwise limiting or to impose or imply any serial or numerical limitations or orderings. Similarly, words such as “particular”, “specific”, “certain”, and “given”, in the description and claims, if used, are to distinguish or identify, and are not intended to be otherwise limiting.

For the purposes of this disclosure, including the claims, the terms “multiple” and “plurality” mean “two or more,” and include the case of “two”. Thus, for example, the phrase “multiple ABCs” means “two or more ABCs” and includes “two ABCs”. Similarly, for example, the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, for example, “about 3” or “approximately 3” shall also cover exactly 3, and “substantially constant” shall also cover exactly constant.

For the purposes of this disclosure, unless stated otherwise, the terms “about” or “approximately” refer to a value that is within 10% above or below the value being described.

For the purposes of this disclosure, including the claims, singular forms of terms are to be construed as also including the plural form and vice versa, unless the context indicates otherwise. Thus, it should be noted that for the purposes of this disclosure, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

For the purposes of this disclosure, the terms “administration” or “administering” refer to a method of giving a dosage of a compound or pharmaceutical composition to a subject.

For the purposes of this disclosure, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (for example, by reducing one or more symptoms of a neurological disorder). The term “therapeutic” includes the effect of mitigating deleterious clinical effects of certain processes (i.e., consequences of the process, rather than the symptoms of processes).

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“for example,”), and the like, is merely intended to better illustrate the invention and does not indicate a limitation on the scope of the invention unless specifically so claimed.

While the invention has been described in connection with what is presently considered to be the most practical, and embodiments thereof are further described in the examples below, it is to be understood that the invention is not to be limited to the disclosed embodiment, but on the contrary, is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

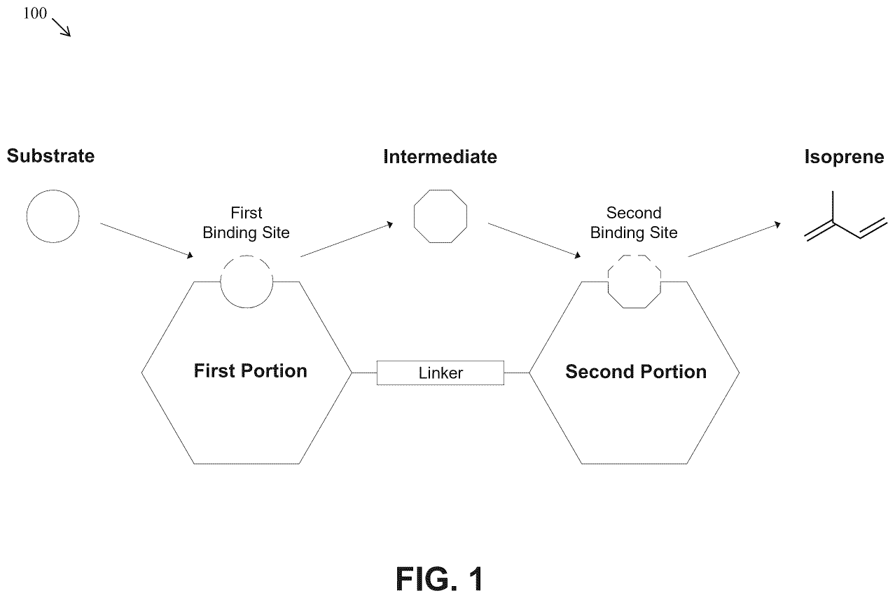

Referring now to FIG. 1, an exemplary embodiment of a genetically engineered peptide superstructure for production of isoprene and its working principle is illustrated as scheme 100. For the purposes of this disclosure, a “peptide superstructure” is a peptide, or a group of peptides assembled together, that contains multiple active sites for performing one or more biological functions. In some cases, biological function may include a catalytic function. For the purposes of this disclosure, a “catalytic function” is the ability of a chemical to accelerate a chemical reaction by functioning as a catalyst. Specifically, a catalyst performs its catalytic function by lowering at least an activation barrier along a reaction coordinate and increasing at least a rate constant associated with the at least an activation barrier. In some cases, to perform a catalytic function, a catalyst may first be consumed by one or more reactants to form one or more intermediates, then be regenerated as the one or more intermediates are converted to one or more products. In some cases, one or more reactants may bind to a catalyst, participate in a chemical reaction, then dissociate from the catalyst as one or more products.

With continued reference to FIG. 1, in one or more embodiments, peptide superstructure may include an enzyme. For the purposes of this disclosure, an “enzyme” is a biological catalyst, often a protein, having a three-dimensional structure specifically tailored for fitting a substrate or reactant and catalyzing a chemical reaction therefrom. Enzymes are often characterized by their high catalytic activity, high specificity toward substrates, and sensitivity to environmental factors such as temperature and pH. A substrate may interact with an enzyme (and its active/binding site) via a lock-and-key mechanism or an induced fit. The catalytic function of a catalyst or enzyme may be described using mathematical tools such as Arrhenius equation, Eyring equation, Michaelis-Menten equation, Lineweaver-Burk equation, among others, as deemed suitable by a person of ordinary skill in the art upon reviewing the entirety of this disclosure.

With continued reference to FIG. 1, peptide superstructure includes a first portion and a second portion. For the purposes of this disclosure, a “portion” of a peptide, peptide superstructure, or enzyme is a unitary structure or substructure containing one or more active sites to which a chemical may bind to initiate, terminate, enhance, inhibit, or otherwise modulate one or more biological functions. In some cases, active site may include a catalytically active site where a reactant or substrate may be converted to a product or metabolite at an accelerated rate, as described above. In one or more embodiments, first portion may include at least a functional fragment of a first enzyme, whereas second portion may include at least a functional fragment of a second enzyme distinct from the first enzyme. For the purposes of this disclosure, a “functional fragment” of an enzyme is a part of the enzyme that includes at least a catalytically active site and therefore preserves at least a catalytic function of the enzyme. In some cases, such functional fragments may be created by truncating or simplifying an enzyme to eliminate peripheral, nonessential structures to improve one or more aspects of its properties, such as without limitation solubility. In some cases, first portion and second portion may be fused into a single enzyme that contains multiple active sites.

With continued reference to FIG. 1, in one or more embodiments, second portion may be covalently linked to first portion through a linker. For the purposes of this disclosure, a “linker” is an extended chemical structure that provides a connection between two or more chemical structures using chemical bonds, thereby bringing the two or more chemical structures in proximity with one another. A linker may perform its linking function using any type of intra- and/or intermolecular interaction deemed suitable by a person of ordinary skill in the art, upon reviewing the entirety of this disclosure, such as without limitation covalent bonds including coordinate covalent bonds, electrostatic interactions, van der Waals/hydrophobic contact, hydrogen bonds, among others. In some cases, linker may include a peptide linker. In some cases, first and second portion may be linked together by transcribing two adjacent genes encoding two enzymes in a continuous DNA sequence, consistent with details described in this disclosure, wherein the two adjacent genes are separated by a spacer gene that encodes a peptide linker, such as a GGGS linker (SEQ ID NO: 27), a GSGGGGS linker (SEQ ID NO: 28), a GSGEAAAKEAAAK linker (SEQ ID NO: 29), or the like, depending on the exact length and/or confirmation needed. In some cases, linker may include a linker of an angular or branched geometry to bring first portion and second portion within proximity of one another. In some cases, first and second portion may be linked via post-translational and/or post-synthetic modifications to yield peptide superstructure. A “linker,” as used in this disclosure, is a nucleotide sequence that encodes a peptide sequence that is inserted between two polynucleotide sequences encoding protein domains or enzymes, such that upon expression the resulting polypeptide contains both domains separated by the linker sequence. A “flexible linker,” as used in this disclosure, is a linker that permits high conformational freedom and allow the fused domains to move relative to one another with minimal steric hindrance. A flexible linker may include amino acid residues such as glycine and serine. For example and without limitation, a glycine-serine (GS) dipeptide sequence may function as a flexible linker in a fusion enzyme construct. A “rigid linker,” as used in this disclosure, is a linker that may include amino acid residues that favor ordered secondary structure and resist random coil conformations, thereby maintaining a more fixed distance and orientation between fused domains. For example and without limitation, an alanine-glutamate-lysine (AEK) tripeptide sequence, or related repeats such as EAAAK, may function as a rigid linker that stabilizes an α-helical conformation and limits conformational flexibility. In one or more embodiments, flexible linkers may be used where independent folding or conformational mobility of enzyme domains is desirable, whereas rigid linkers may be used to preserve spatial separation or to orient active sites in a defined geometry, thereby reducing steric interference or promoting desired catalytic efficiency.

With continued reference to FIG. 1, first portion includes a first binding site configured to catalyze a first chemical reaction that converts a substrate to an intermediate. For the purposes of this disclosure, an “intermediate” is a chemical that forms as a product in a first reaction step and is consumed as a reactant in a second reaction step, with no net accumulation. In one or more embodiments, first portion may include at least a functional fragment of isopentenyl diphosphate (IPP) isomerase (idi). Accordingly, in one or more embodiments, substrate may include IPP. In one or more embodiments, intermediate may include dimethylallyl pyrophosphate (DMAPP). IPP and DMAPP may be produced via the methylerythritol phosphate (MEP) and/or the mevalonate (MVA) pathways, as described in detail below in this disclosure.

With continued reference to FIG. 1, second portion includes a second binding site configured to catalyze a second chemical reaction converting intermediate to isoprene. In one or more embodiments, second portion may include at least a functional fragment of isoprene synthase (IspS). IspS is capable of catalyzing a conversion from DMAPP to isoprene. Additional details will be provided below in this disclosure.

With continued reference to FIG. 1, second binding site is located in proximity to first binding site. In some cases, second binding site may be 1 nanometer-1 micrometer away from first binding site. As nonlimiting examples, second binding site may be separated from first binding site by 1 nanometer to 2 nanometers, 2 nanometers to 3 nanometers, 3 nanometers to 4 nanometers, 4 nanometers to 5 nanometers, 5 nanometers to 6 nanometers, 6 nanometers to 7 nanometers, 7 nanometers to 8 nanometers, 8 nanometers to 9 nanometers, 9 nanometers to 10 nanometers, 10 nanometers to 20 nanometers, 20 nanometers to 50 nanometers, 50 nanometers to 100 nanometers, 100 nanometers to 200 nanometers, 200 nanometers to 300 nanometers, 300 nanometers to 400 nanometers, 400 nanometers to 500 nanometers, 500 nanometers to 600 nanometers, 600 nanometers to 700 nanometers, 700 nanometers to 800 nanometers, 800 nanometers to 900 nanometers, or 900 nanometers to 1 micrometer. Such proximity may facilitate an immediate transformation of intermediate to isoprene that bypasses potential side reactions, thereby improving the yield of isoprene. Additional details will be provided below in this disclosure.

With continued reference to FIG. 1, for the purposes of this disclosure, “isoprene”, also known as 2-methyl-1,3-butadiene, is a volatile organic compound with the chemical formula C₅H₈, a structural formula of CH₂═CHC(CH₃)═CH₂, and a normal boiling point of 34° C. Isoprene may be considered a “hemiterpene” (i.e., half of a terpene), and the details of terpenes will be described below in this disclosure. Isoprene is a key building block for synthesizing natural rubber and various other polymers. Additionally, isoprene may be used in the manufacture of synthetic rubber, adhesives, and elastomers, as well as in the production of pharmaceuticals and other specialty chemicals. Isoprene may be produced naturally by plants and trees, particularly in large amounts by the rubber tree (i.e., Hevea brasiliensis). It may also be produced industrially through the thermal cracking of petroleum-derived naphtha or, in some cases, through biotechnological methods using genetically engineered microbes, as described in this disclosure. In some cases, isoprene may be synthesized using industrial organic chemistry, via synthetic routes such as an acetone/acetylene route, a propylene dimer route, an isoamylene route, an isopentane route, or an isobutylene/formaldehyde route, among others.

With continued reference to FIG. 1, for the purposes of this disclosure, a “terpene” is an organic compound with the general molecular formula (C₅H₈)ₙ, often characterized by a structure that contains repeating isoprene units. Terpenes are produced predominantly by plants, particularly conifers, and certain insects, and may be involved in various biological functions, including defense mechanisms and communication. Terpenes may be classified as monoterpenes (n=2), sesquiterpenes (n=3), diterpenes (n=4), and higher terpenes (n>4), based on the number of isoprene units they contain. Monoterpenes (C₁₀H₁₆) may include without limitation limonene found in citrus peels, myrcene in hops and lemongrass, pinene in pine resin, linalool in lavender, and geraniol in rose oil, among others. Sesquiterpenes (C₁₅H₂₄) may include without limitation farnesene in apple coatings, humulene in hops, bisabolol in chamomile, caryophyllene in black pepper, and nerolidol in ginger and tea tree oil. Diterpenes (C₂₀H₃₂) may include without limitation taxadiene from yew trees, gibberellins as plant hormones, phytol in chlorophyll, steviol in Stevia leaves, and retinol (Vitamin A1) derived from β-carotene. Higher terpenes may include without limitation squalene (C₃₀H₅₀) in shark liver oil, lanosterol (C₃₀H₅₀O) in wool grease, tetraterpenes or tetraterpenoids including carotenoids (C₄₀H₅₆) such as beta-carotene in carrots, and rubber (polyterpene) from latex. Terpenes may serve as a biochemical precursor for a wide array of natural products, including essential oils, resins, and various pharmacologically active compounds, making them valuable in industries such as pharmaceuticals, perfumery, and agriculture. Isoprene belongs to a broader class of molecules called isoprenoids, and similarly, terpenes belong to a broader class of molecules called terpenoids, though it is worth noting that these terminologies may often be used interchangeably. An isoprenoid may include a molecule that is derived from isoprene, and similarly, a “terpenoid” may include a molecule that is derived from a terpene, consistent with details described above. Isoprenoids and terpenoids include a large and diverse class of naturally occurring organic compounds. Isoprenoids/terpenoids may also be synthesized through either the MEP pathway or the MVA pathway, as described in detail above. Isoprenoids/terpenoids may play essential roles in various biological processes, including without limitation cellular membrane structure (as cholesterol), photosynthesis (as carotenoids), and growth regulation (as gibberellins). Isoprenoids/terpenoids may also be used in pharmaceuticals, fragrances, and biofuels due to their diverse chemical properties and biological activities.

With continued reference to FIG. 1, it is worth noting that the invention described in this disclosure is not limited to production of isoprene only. As a nonlimiting example, the precursor for producing isoprene, such as without limitation IPP and/or DMAPP, may also be a building block for other isoprenoids, terpenes, or terpenoids; therefore, an accelerated biosynthesis of isoprene may contribute to an increase in yield for producing these isoprenoids, terpenes, or terpenoids. Specifically, in some cases, products of the MVA pathway such as DMAPP and IPP may be converted to geranyl diphosphate (GPP), which may be further converted to monoterpenes, etc. In some cases, products of the MVA pathway such as DMAPP and IPP may be converted to farnesyl diphosphate (FPP), which may be further converted to sesquiterpenes, triterpenes, and/or carotenoids, among others. A person of ordinary skill in the art, upon reviewing the entirety of this disclosure, will be able to recognize how the invention described herein may be extended to other related applications beyond isoprene synthesis.

With continued reference to FIG. 1, at least part of peptide superstructure may be produced by a genetically engineered microbe. For the purposes of this disclosure, a “genetically engineered microbe” is a microbe whose genetic material has been manipulated to alter one or more of its hereditary traits. Such manipulation may include without limitation inserting, deleting, or otherwise modifying one or more specific DNA sequences; as a result of such manipulation, a genetically modified microbe may exhibit one or more different traits in its structure, function, or the like, compared to its naturally occurring (i.e., wild-type) counterparts. For the purposes of this disclosure, a “microbe” is a microscopic organism, including bacteria, archaea, fungi, protozoa, viruses, and/or the like, that is capable of being utilized in one or more aspects of a biotechnological application. A microbe may be characterized by its ability to perform certain biological processes, such as without limitation fermentation, gene expression, and metabolite production, among others. A microbe may be harnessed for purposes such as without limitation recombinant protein production, bioremediation, synthesis of pharmaceuticals, and development of biofuels, among others, due to their diverse metabolic capabilities and ease of genetic manipulation. For the purposes of this disclosure, a “suitable condition” is an environmental condition or factor suitable for the growth and/or replication of a microbe. In some cases, a suitable condition may vary from one type of microbe to another. A suitable condition may include without limitation a suitable temperature/temperature range, a suitable pressure/pressure range, a suitable pH or pH range, a suitable ionic strength/range of ionic strength, a suitable osmolarity/range of osmolarity, a suitable osmotic pressure/range of osmotic pressure, a suitable concentration/concentration range of one or more nutrients, and/or a suitable level of metabolic waste/metabolites, among others. A person of ordinary skill in the art, upon reviewing the entirety of this disclosure, will be able to identify suitable conditions specific to one or more microbes described herein.

With continued reference to FIG. 1, in one or more embodiments, genetically engineered microbe may include bacteria or fungi. In one or more embodiments, genetically engineered microbe may include Escherichia including Escherichia Coli (or E. coli), Lactococcus including Lactococcus lactis, Vibrio including Vibrio natriegens (or V. natriegens), Saccharomyces including Saccharomyces cerevisiae (or S. cerevisiae), Aspergillus including Aspergillus niger, Aspergillus nidulans, and/or Aspergillus oryzae, Yarrowia including Yarrowia lipolytica (Y. lipolytica), Bacillus including Bacillus subtilis (B. subtilis), Clostridium including Clostridium ljungdahlii (C. ljungdahlii), and/or Pseudomonas including Pseudomonas putida (P. putida), among others. In one or more embodiments, different E. coli may be used in cloning and expression. For example and without limitation, E. coli may include BL21(DE3), C41 (DE3), C43 (DE3), BL21-AI, Origami (DE3), Rosetta-gami (DE3), SHuffle T7, Tuner strains (lacY mutants), Mutant56 (DE3), DH5α, JM109, XL1-Blue, TOP10, HB101, MG1655, W3110, DH1, RV308, HMS174, LS5218.

With continued reference to FIG. 1, for the purposes of this disclosure, Escherichia coli (E. coli) is a widely studied bacterium that serves as a crucial model organism in biotechnology and molecular biology. E. coli is a gram-negative, rod-shaped bacterium naturally found in the intestines of humans and other warm-blooded animals. The simplicity, well-characterized genetics, and rapid growth of E. coli, among other traits, make it a suitable host for genetic engineering applications. It is commonly used for producing recombinant proteins, plasmid cloning, and various metabolic engineering processes.

With continued reference to FIG. 1, for the purposes of this disclosure, Lactococcus is a genus of Gram-positive, facultatively anaerobic bacteria that belongs to the family Streptococcaceae. Lactococcus bacteria are non-spore-forming, usually non-motile, and commonly occur as spherical or ovoid cells that often form chains or pairs. Lactococcus species are lactic acid bacteria (LAB), i.e., they are capable of fermenting sugars to produce lactic acid, a process that may be useful in various food fermentation systems. Lactococcus species such as without limitation Lactococcus lactis are widely used in the dairy industry, particularly in the production of fermented dairy products like cheese and buttermilk, wherein the lactose in milk is converted into lactic acid, which lowers the pH, inhibits the growth of harmful microorganisms, and contributes to the texture and flavor of the final product. Beyond food fermentation, Lactococcus may be used in biotechnology for producing recombinant proteins, such as without limitation enzymes and therapeutic proteins, due to its relatively simple genetic system. Additionally, some Lactococcus strains may have probiotic potential and/or an ability to produce bioactive compounds, offering applications in food preservation and health supplements.

With continued reference to FIG. 1, for the purposes of this disclosure, Vibrio natriegens (V. natriegens) is a fast-growing, gram-negative marine bacterium. V. natriegens has recently gained prominence in biotechnology due to its exceptionally rapid doubling time of less than 10 minutes under suitable conditions. The fast growth, genetic tractability, high transformation efficiency, and ability to thrive in simple media of V. natriegens, among other traits, make it a suitable organism for various applications, including synthetic biology, recombinant protein production, metabolic engineering, and high-throughput screening, among others.

With continued reference to FIG. 1, for the purposes of this disclosure, Saccharomyces cerevisiae (S. cerevisiae) is a unicellular eukaryote, commonly known as brewer's yeast or baker's yeast, that plays a pivotal role in biotechnology due to its well-characterized genetics and ease of manipulation. S. cerevisiae has been instrumental in winemaking, baking, and brewing since ancient times and is a model system extensively used for recombinant DNA technology, metabolic engineering, and synthetic biology applications. Its ability to perform post-translational modifications similar to higher eukaryotes makes it suitable for producing complex proteins and biopharmaceuticals. Additionally, S. cerevisiae may be employed in biofuel production, fermentation processes, and various industrial biotechnology applications.

With continued reference to FIG. 1, for the purposes of this disclosure, Aspergillus is a genus of filamentous fungi. Aspergillus is characterized by its ability to produce a wide array of enzymes, organic acids, and secondary metabolites, making it a suitable host for production of biofuels, pharmaceuticals, and food products. Several genetic manipulation techniques have been established for Aspergillus. Certain Aspergillus species, such as Aspergillus niger, Aspergillus nidulans, and Aspergillus oryzae, may be used for recombinant protein production and metabolic engineering due to their efficient protein secretion systems and robust metabolic capabilities. Additionally, and/or alternatively, other filamentous fungi such as without limitation Fusarium and/or Neurospora may be used.

With continued reference to FIG. 1, for the purposes of this disclosure, Yarrowia lipolytica (Y. lipolytica) is a species of yeast belonging to genus Yarrowia and the family Dipodascaceae. It is known for its ability to utilize a wide range of carbon sources, particularly lipids, which makes it a useful organism in biotechnological applications. Yarrowia lipolytica may be used in the production of various bioproducts, including single-cell oils, enzymes, and organic acids. Yarrowia lipolytica is also notable for its potential use in bioremediation, as it may degrade environmental pollutants such as hydrocarbons and fatty acids. Yarrowia lipolytica has a well-studied genetic system, allowing for genetic modifications and metabolic engineering to enhance its production capabilities. Additionally, Yarrowia lipolytica is recognized for its lack of toxicity in food and industrial applications, making it an attractive candidate for use in various biotechnological processes.

With continued reference to FIG. 1, for the purposes of this disclosure, Bacillus subtilis (B. subtilis), is a Gram-positive, rod-shaped bacterium commonly found in soil and the gastrointestinal tracts of ruminants and humans. Bacillus subtilis is known for its ability to form endospores, which allow it to survive in harsh environmental conditions. Bacillus subtilis is widely studied for its role in various applications, including biotechnology, agriculture, and food production. Bacillus subtilis serves as a model organism for laboratory studies due to its genetic tractability and well-characterized physiology. Additionally, Bacillus subtilis may be utilized as a probiotic in animal feed and/or as a biocontrol agent in agriculture, promoting plant growth and/or protecting crops from pathogens. Its capability of producing enzymes, antibiotics, and/or other bioactive compounds further underscores its significance in various industrial processes.

With continued reference to FIG. 1, for the purposes of this disclosure, Clostridium ljungdahlii (C. ljungdahlii) is a gram-positive, anaerobic, rod-shaped bacterium that is part of the genus Clostridium. It is notable for its ability to perform autotrophic growth by utilizing a mixture of carbon monoxide (CO), carbon dioxide (CO₂), and hydrogen (H₂) (i.e., syngas) as sole carbon and energy sources, through a process known as the Wood-Ljungdahl pathway (acetyl-CoA pathway). Clostridium ljungdahlii is capable of converting syngas into value-added products, such as without limitation acetone, butanol, ethanol (ABE), and acetic acid, making it an important organism for industrial biotechnology applications, particularly in gas fermentation (for example, syngas fermentation) and biofuel production. Clostridium ljungdahlii holds promise for producing renewable fuels and chemicals from waste gases, such as those generated by industrial processes or gasified biomass. Specifically, an advantage of Clostridia as a host organism may derive from their ability to employ diverse carbon sources, including mono-, oligo- and polysaccharides that would be found in waste products, making the fermentation of industrial, agricultural, and waste products conceivable.

With continued reference to FIG. 1, for the purposes of this disclosure, Pseudomonas putida (P. putida) is a gram-negative, rod-shaped, aerobic bacterium belonging to the genus Pseudomonas. Pseudomona sputida is commonly found in soil, water, and various environments where organic compounds are present. Pseudomonas putida is notable for its metabolic versatility, which allows it to degrade a wide range of organic compounds including without limitation hydrocarbons, aromatic compounds, and/or environmental pollutants such as without limitation toluene and naphthalene, making it an important organism for environmental bioremediation. Additionally, Pseudomona sputida may be used in various industrial and biotechnological applications due to its ability to produce valuable compounds and its resilience under harsh conditions. Pseudomona sputida may also be used to produce chemicals, such as biofuels, bioplastics, and other value-added compounds, through microbial fermentation and/or genetic engineering.

With continued reference to FIG. 1, in some cases, creating a genetically engineered microbe may involve using recombinant DNA technology, CRISPR-Cas9, gene cloning, and/or other molecular biology techniques to achieve desired phenotypic changes. For the purposes of this disclosure, “recombinant DNA technology” is a type of technology that involves a manipulation of DNA sequences to create new genetic combinations not found in nature. Recombinant DNA technology may include techniques such as gene cloning, insertion of DNA fragments from one organism into another, and/or use of vectors such as plasmids to transfer a genetic material. Recombinant DNA technology may be used to express new traits or produce biological products such as proteins, enzymes, and hormones and may play a pivotal role in fields such as genetic engineering, biotechnology, and pharmaceutical development.

With continued reference to FIG. 1, for the purposes of this disclosure, a “plasmid” is a circular, double-stranded DNA molecule distinct from a cell's chromosomal DNA and capable of autonomous replication. A plasmid may be used as a vector for insertion, expression, and propagation of foreign genes within a host organism. Such vectors may include specific sequences for an origin of replication, selectable markers, and cloning sites, enabling manipulation and study of genetic material for applications in research, biotechnology, and therapeutic development.

With continued reference to FIG. 1, for the purposes of this disclosure, “CRISPR-Cas9” is a genetic engineering tool, derived from a bacterial immune defense mechanism, that includes Clustered Regularly Interspaced Short Palindromic Repeats (CRISPR) sequences and the CRISPR-associated protein 9 (Cas9) enzyme and allows for precise, targeted modification of DNA within an organism. CRISPR-Cas9 may be programmed with a guide RNA to target a specific DNA sequence, enabling Cas9 enzyme to create double-strand breaks at one or more precise locations, thereby facilitating insertion, deletion, or modification of genetic material.

With continued reference to FIG. 1, genetically engineered microbe includes a heterologous nucleic acid construct. For the purposes of this disclosure, a “heterologous” nucleic acid construct is a nucleic acid construct that originates from a different species or is artificially synthesized and introduced into a host organism. A heterologous nucleic acid construct may be inserted into a host genome or expressed in a host cell to produce proteins, confer new traits, and/or study gene functions. A heterologous nucleic acid may include genes, regulatory elements, or other sequences that are not naturally found in the host organism's genome. For the purposes of this disclosure, a “nucleic acid construct” is a nucleic acid sequence that encodes a peptide and is capable of being expressed to synthesize the peptide. In other words, a nucleic acid construct includes a coding sequence that encodes a peptide and one or more regulatory elements that regulate the expression of the coding sequence. Exemplary embodiments of a regulatory element may include a promoter, an enhancer, a silencer, an insulator, an operator, and a response element, among others. For the purposes of this disclosure, a “promoter” is a DNA sequence where an RNA polymerase binds to initiate transcription. For the purposes of this disclosure, an “enhancer” is a distant element of DNA sequence that increases transcription rates by interacting with a promoter via DNA looping. For the purposes of this disclosure, a “silencer” is a DNA sequence that represses transcription when bound by specific proteins. For the purposes of this disclosure, an “insulator” is a DNA sequence that prevents the interaction between one or more enhancers and one or more promoters of neighboring genes. For the purposes of this disclosure, an “operator” is a DNA segment that regulates the transcription of adjacent genes. For the purposes of this disclosure, a response element is a DNA sequence that responds to external signals, allowing genes to be turned on or off in response to environmental changes. These regulatory elements may work together to ensure precise gene expression necessary for proper cellular function.

With continued reference to FIG. 1, in one or more embodiments, nucleic acid construct may include one or more operons. For the purposes of this disclosure, an “operon” is a functioning unit of DNA containing a cluster of genes under the control of a single promoter. It is commonly found in prokaryotes such as bacteria. These genes are transcribed together into a single messenger RNA strand and typically encode proteins that work together in a specific biological pathway. An operon may include a regulatory element, such as an operator as described above, where an activator or repressor protein may bind to increase or inhibit transcription. An operon may include one or more regulatory genes that encode one or more such activator or repressor proteins. A nonlimiting example of operon may include the lac operon, which regulates lactose metabolism in E. coli. In one or more embodiments, lac operon may provide a regulatory framework widely adopted for recombinant expression in E. coli. By placing a heterologous gene under the control of a lac promoter or hybrid promoter (trc, tac, lacUV5), expression may be repressed by LacI protein until induction with IPTG. In engineered strains such as BL21(DE3), induction with IPTG may trigger production of T7 RNA polymerase from a lac-regulated cassette, which in turn drives high-level expression of a target gene cloned under a T7 promoter.

With continued reference to FIG. 1, in one or more embodiments, a vector may be used to genetically engineer a microbe. a “vector” may be any of a number of nucleic acid molecules, viruses, or portions thereof that are capable of mediating entry of, for example, transferring, transporting, or otherwise delivering, a nucleic acid of interest between different genetic environments or into a cell. A nucleic acid of interest may be linked to, or inserted into, the vector using, for example, restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, or transposons (for example, the Sleeping Beauty transposon). Plasmid vectors typically include an origin of replication, for example, replication in prokaryotic cells. A plasmid may include part or all of a viral genome, for example, a viral promoter, enhancer, processing or packaging signals, or sequences sufficient to give rise to a nucleic acid that can be integrated into the host cell genome and/or to give rise to infectious virus. Viruses or portions thereof that can be used to introduce nucleic acids into cells may be referred to as viral vectors. Viral vectors include, for example, adenoviruses, adeno-associated viruses, retroviruses (such as lentiviruses or gamma retroviruses), vaccinia virus and other poxviruses, herpesviruses (such as herpes simplex virus), and others. Viral vectors may or may not contain sufficient viral genetic information for production of infectious virus when introduced into host cells, i.e., viral vectors may be replication-competent or replication-defective. In one or more embodiments, for example where sufficient information for production of infectious virus is lacking, it may be supplied by a host cell or by another vector introduced into the cell if production of virus is desired. In one or more embodiments, such information is not supplied if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, proteins that increase or decrease resistance or sensitivity to antibiotics or other agents (such as a protein conferring resistance to puromycin, hygromycin, or blasticidin), enzymes whose activities are detectable by assays known in the art (for example, β-galactosidase or alkaline phosphatase), and proteins or RNAs that detectably affect the phenotype of cells that express them (for example, fluorescent proteins). Vectors often include one or more appropriately positioned sites for restriction enzymes, which may be used to facilitate insertion into the vector of a nucleic acid, for example a nucleic acid to be expressed. An expression vector is a vector into which a desired nucleic acid has been inserted or may be inserted such that it is operably linked to regulatory elements (also termed “regulatory sequences,” “expression control elements,” or “expression control sequences”) and may be expressed as an RNA transcript, for example an mRNA that can be translated into protein or a noncoding RNA such as an shRNA or miRNA precursor. Expression vectors include regulatory sequence(s), for example expression control sequences, sufficient to direct transcription of an operably linked nucleic acid under at least some conditions; other elements required or helpful for expression may be supplied by, for example, the host cell or by an in vitro expression system. Such regulatory sequences typically include a promoter and may include enhancer sequences or upstream activator sequences. In some embodiments, a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired, may be contained in an inserted nucleic acid, or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species, for example a mammalian species, or in a desired cell type. A vector may contain a promoter capable of directing expression in mammalian cells, such as a suitable viral promoter, for example from cytomegalovirus (CMV), retrovirus, simian virus (for example SV40), papillomavirus, herpesvirus or other virus that infects mammalian cells, or a mammalian promoter from, for example, a gene such as EF1alpha, ubiquitin (such as ubiquitin B or C), globin, actin, phosphoglycerate kinase (PGK), or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase I (a “pol I promoter”), for example a promoter for transcription of ribosomal RNA (other than 5S rRNA) may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase III (a “pol III promoter”), for example a promoter for transcription of U6, H1, 7SK or tRNA or a functional variant thereof, is used. One of ordinary skill in the art will select an appropriate promoter for directing transcription of a sequence of interest. Examples of expression vectors that may be used in mammalian cells include, for example, the pcDNA vector series, pSV2 vector series, pCMV vector series, pRSV vector series, pEFI vector series, and Gateway® vectors. Examples of virus vectors that may be used in mammalian cells include, for example, adenoviruses, adeno-associated viruses, poxviruses such as vaccinia viruses and attenuated poxviruses, retroviruses (for example lentiviruses), Semliki Forest virus, Sindbis virus, and others. In some embodiments, regulatable (for example, inducible or repressible) expression control element(s), for example a regulatable promoter, is/are used so that expression can be regulated, for example turned on or increased or turned off or decreased. For example, the tetracycline-regulatable gene expression system (Gossen & Bujard, Proc. Natl. Acad. Sci. 89:5547-5551, 1992) or variants thereof (see, for example, Urlinger, S, et al. (2000), Zhou, X., et al (2006)) can be employed to provide inducible or repressible expression. Other inducible/repressible systems may be used in various embodiments. For example, expression control elements that can be regulated by small molecules such as artificial or naturally occurring hormone receptor ligands (for example steroid receptor ligands such as naturally occurring or synthetic estrogen receptor or glucocorticoid receptor ligands), tetracycline or analogs thereof, or metal-regulated systems (for example the metallothionein promoter) may be used in certain embodiments. In some embodiments, tissue-specific or cell type-specific regulatory element(s) may be used, for example to direct expression in one or more selected tissues or cell types.

With continued reference to FIG. 1, in one or more embodiments, golden gate assembly may be used as a molecular cloning method to join multiple DNA fragment. “Golden Gate Assembly,” as used in this disclosure, is a molecular cloning technique in which multiple DNA fragments may be directionally joined in a single reaction. For example and without limitation, DNA fragment may be joined using a Type IIS restriction enzyme and a DNA ligase. A Type IIS restriction enzyme may include, without limitation, BsaI, BsmBI, or BbsI, which recognizes a specific nucleotide sequence but cleaves the DNA at a defined distance outside of the recognition site. As a result, cleavage may generate a set of user-defined single-stranded overhangs that may be designed to anneal only in a predetermined order, allowing directional assembly of multiple DNA fragments into a vector. A simultaneous action of digestion and ligation in a one-pot reaction may permit repeated cleavage of incorrectly ligated intermediates, while correctly ligated products may accumulate since the recognition sites are removed from the final construct. Thus, Golden Gate Assembly may provide scarless assembly without unwanted sequences remaining at the ligation junction.

With continued reference to FIG. 1, “domesticated Golden Gate Assembly,” as used in this disclosure, refers to a Golden Gate Assembly process in which all internal recognition sites for the Type IIS restriction enzyme are removed from the DNA fragments and vector backbone prior to assembly. Domestication may include introducing silent mutations within coding regions or other sequence modifications that do not alter the encoded protein or functional regulatory element, thereby preventing undesired cleavage during the assembly process and enabling efficient and accurate construction of recombinant DNA.

With continued reference to FIG. 1 restriction enzymes, which may be used to facilitate insertion into the vector of a nucleic acid. For example and without limitation, restriction enzymes may include Type IP (sticky 5′ overhangs): EcoRI, BamHI, HindIII, XhoI, SalI, NheI, XbaI, SpeI, BglII, NcoI, NdeI, AgeI, NotI, AvrII, ClaI, MluI; Type IP (sticky 3′ overhangs): PstI, KpnI, SacI, SphI, ApaI, NsiI; Type IP (blunt): EcoRV, SmaI, PvuII, StuI, DraI, ScaI; Type IIS/IIG: BsaI, BsmBI (Esp3I), BbsI, SapI (BspQI), AarI, FokI, BtgZI; and Homing endonucleases: I-SceI, I-CeuI, PI-SceI, PI-PspI.

With continued reference to FIG. 1, nucleic acid construct encodes at least a portion of peptide superstructure, consistent with details described above.

With continued reference to FIG. 1, in one or more embodiments, genetically engineered microbe may include a Crabtree-negative microbe. For the purposes of this disclosure, a “Crabtree-negative” microbe is a microbe that that does not exhibit the Crabtree effect. A Crabtree-negative microbe may maintain aerobic respiration and efficient ATP production via the tricarboxylic acid cycle and oxidative phosphorylation under high glucose conditions. Such characteristic makes Crabtree-negative microbes potentially valuable for biotechnological applications that require efficient biomass yield and metabolic productivity without the inhibitory effects of fermentation byproducts. Common Crabtree-negative microbes include without limitation E. coli, Pseudomonas aeruginosa (P. aeruginosa), Bacillus subtilis (B. subtilis), Corynebacterium glutamicum (C. glutamicum), and Saccharomyces kluyveri (S. kluyveri), among others. In contrast, a “Crabtree-positive” microbe is a microbe that exhibits the Crabtree effect. Common Crabtree-positive microbes include without limitation S. cerevisiae, Saccharomyces pastorianus (lager yeast), and/or Schizosaccharomyces pombe (fission yeast).

With continued reference to FIG. 1, for the purposes of this disclosure, the “Crabtree effect”, named after the English biochemist Herbert Grace Crabtree, is a phenomenon where high glucose concentrations inhibit cellular respiration, leading to fermentation even in the presence of oxygen. The Crabtree effect may offer a microbe certain evolutionary advantage, as the Crabtree effect may allow a microbe to quickly generate ATP and outcompete other microbes in glucose-rich environments. The production of ethanol may also inhibit the growth of competing microbes to provide an additional competitive edge.

With continued reference to FIG. 1, in some cases, genetically engineered microbe may include a diploid strain. For the purposes of this disclosure, a “diploid strain” is a type of organism or cell line that contains two complete sets of chromosomes, one from each parent. In genetic and metabolic engineering, diploid strains may be used to study gene function and regulation, create hybrid organisms, and/or enhance desirable traits. A diploid strain may provide a robust system for complementation tests, enabling researchers to determine the function of specific genes. Additionally, a diploid strain may exhibit increased genetic stability and adaptability, making them valuable for industrial applications such as fermentation and the production of biofuels and pharmaceuticals.

With continued reference to FIG. 1, in some cases, genetically engineered microbe may include balanced MEP and MVA pathways. Additional details will be provided below in this disclosure.

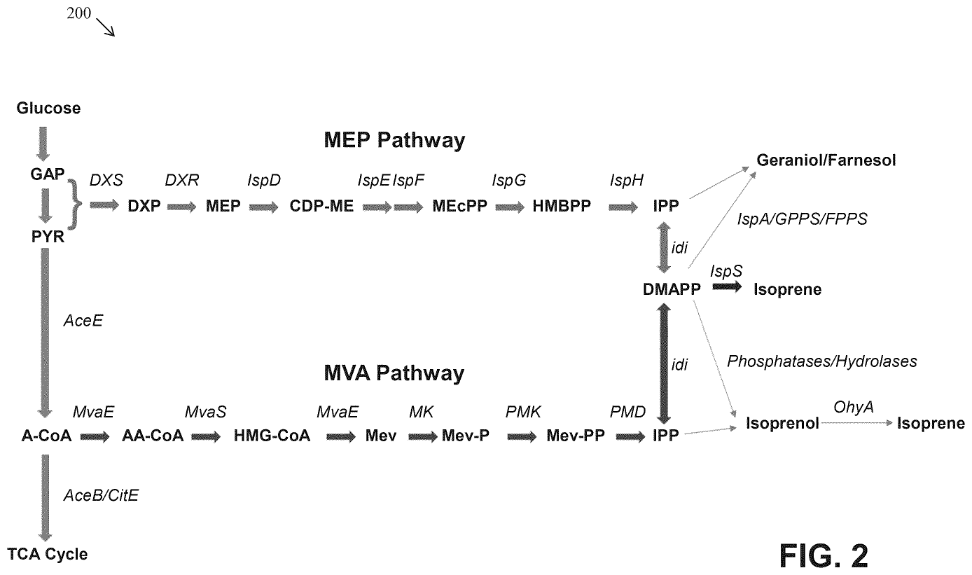

Referring now to FIG. 2, scheme 200 illustrates several metabolic pathways pertaining to biosynthesis of isoprene, highlighting the substrate, product, and/or enzyme (catalyst) associated with each step. Scheme 200 includes part of the central metabolic pathway, i.e., the cellular respiration pathway, wherein glucose is broken down to feed the tricarboxylic acid (TCA) cycle. Conversion of glucose involves several key steps. Initially, glucose (C₆H₁₂O₆) undergoes glycolysis by converting to two molecules of glyceraldehyde 3-phosphate (GAP). Such conversion is catalyzed by enzymes including hexokinase, phosphofructokinase, and aldolase. GAP is then further processed to form pyruvate (C₃H₃O₃⁻), producing a net gain of 2 ATP and 1 NADH. Such conversion is catalyzed by enzymes such as glyceraldehyde 3-phosphate dehydrogenase and pyruvate kinase. Pyruvate is further converted to acetyl coenzyme A (A-CoA), releasing NADH and CO₂. Such conversion is catalyzed by pyruvate dehydrogenase (PDH), which may include pyruvate dehydrogenase E1 component (encoded by gene AceE). A-CoA then enters the TCA cycle (not shown) and participates in reactions catalyzed by enzymes such as without limitation malate synthase (AceB) or ATP citrate lyase (CitE). It is worth noting that glucose is not the only viable carbon source for the invention described in this disclosure; alternatives such as sucrose, biomass, glycerol, ethanol, plant oils, or the like, may be used instead upon strategic engineering of metabolic pathways.

With continued reference to FIG. 2, scheme 200 includes methylerythritol phosphate (MEP) and mevalonate (MVA) pathways for isoprene production. Both the MEP and the MVA pathways are connected to the central metabolic pathway and may lead to production of isoprene, consistent with details described above. For the purposes of this disclosure, the methylerythritol phosphate (MEP) pathway, also known as the non-mevalonate pathway, is a metabolic route for isoprenoid biosynthesis in bacteria, algae, and/or plant plastids. It begins with the formation of 1-deoxy-D-xylulose-5-phosphate (DXP) from pyruvate and glyceraldehyde-3-phosphate (GAP), which is catalyzed by 1-deoxy-D-xylulose-5-phosphate synthase (DXS). This step is one of the rate-determining steps of the MEP pathway. DXP is then converted to 2-C-methyl-D-erythritol-4-phosphate (MEP), which is catalyzed by 1-deoxy-D-xylulose 5-phosphate reductase (DXR). MEP is then converted to 4-diphosphocytidyl-2-C-methyl-D-erythritol (CDP-ME), which is catalyzed by 4-diphosphocytidyl-2-C-methyl-D-erythritol synthase (IspD). CDP-ME is further converted via two steps to 2-C-methyl-D-erythritol-2,4-cyclodiphosphate (MEcPP), which is catalyzed by 4-diphosphocytidyl-2-C-methyl-Derythritol kinase (IspE) and 2-C-methyl-D-erythritol 2,4-cyclodiphosphate synthase (IspF). MEcPP is then reduced to 4-hydroxy-3-methylbut-2-enyl diphosphate (HMBPP), which is catalyzed by (E)-4-Hydroxy-3-methylbut-2-enyl pyrophosphate synthase (IspG). HMBPP is then converted to isopentenyl diphosphate (IPP) and dimethylallyl diphosphate (DMAPP), which is catalyzed by (E)-4-Hydroxy-3-methylbut-2-enyl pyrophosphate reductase (IspH or HMBPP reductase). IPP and DMAPP may establish a chemical equilibrium and interconvert with one another, which is catalyzed by isopentenyl diphosphate isomerase (idi). DMAPP may be converted to isoprene, which is catalyzed by isoprene synthase (IspS). IspS may be derived from plants, as many plants such as kudzu (the Asian vine, Pueraria montana), aspen (Populus tremuloides), and poplar (Populus alba), among others, may naturally produce isoprene by the catalytic elimination of pyrophosphate from DMAPP. Chemical structures of each reactant, intermediate, and product of the MEP pathway are illustrated in Scheme 1 below:

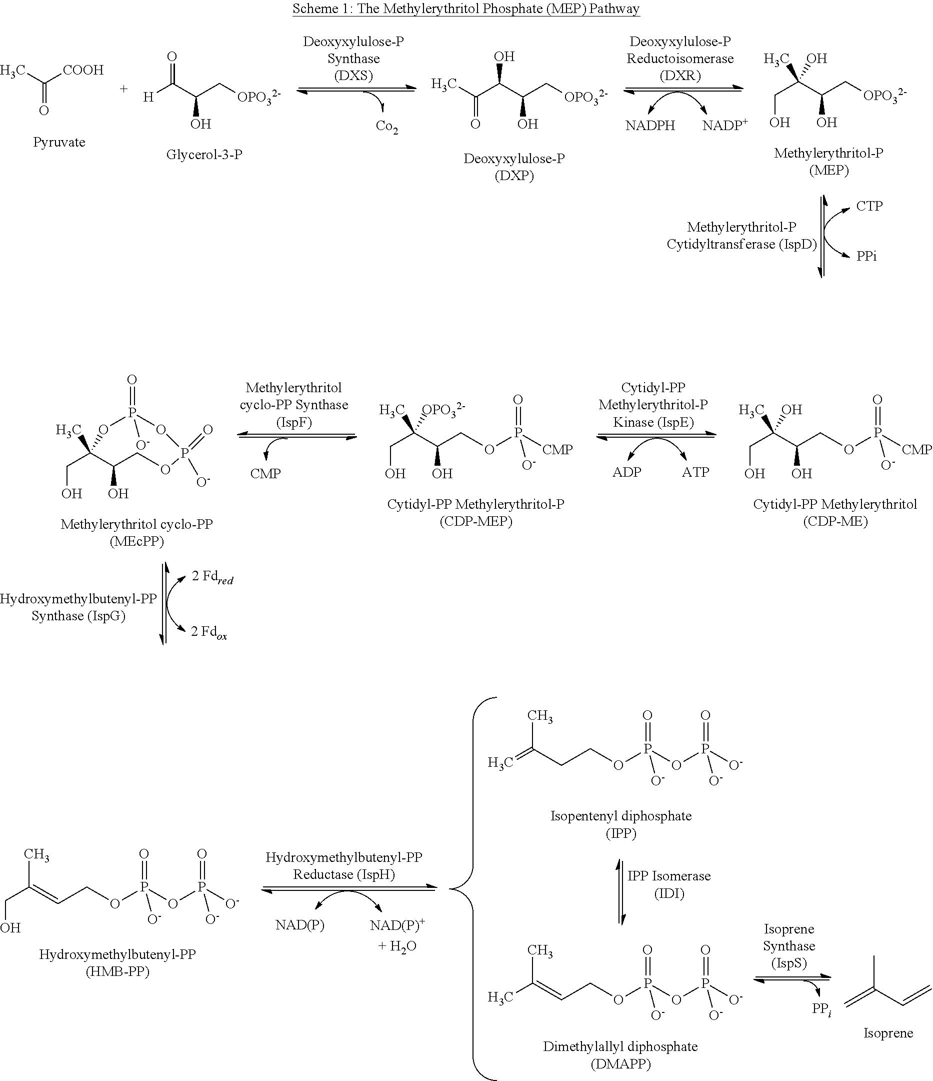

With continued reference to FIG. 2, for the purposes of this disclosure, the mevalonate (MVA) pathway is another metabolic route for isoprenoid biosynthesis, found in archaea, fungi, animals, and some bacteria. The MVA pathway starts with the conversion of A-CoA to acetoacetyl-coenzyme A (AA-CoA), which is catalyzed by acetoacetyl-coenzyme A thiolase or 3-hydroxy-3-methylglutaryl-coenzyme A reductase (MvaE, which may be encoded by gene ERG10). AA-CoA is then converted to 3-hydroxy-3-methylglutaryl-CoA (HMG-CoA), which is catalyzed by HMG-CoA synthase (MvaS, which may be encoded by gene ERG13). HMG-CoA is then reduced to mevalonate (Mev). This step is catalyzed by MvaE, which may be encoded by gene HMG1, HMG2, or HMG-CoA reductase (HMGR). This step is the rate-determining step of the MVA pathway. Mev is phosphorylated to mevalonate-5-phosphate (Mev-P), which is catalyzed by mevalonate kinase (MK), which could be encoded by ERG12. Mev-P is phosphorylated to form mevalonate-5-diphosphate (Mev-PP), which is catalyzed by phosphomevalonate kinase (PMK), which could be encoded by ERG8. Mev-PP is subsequently decarboxylated to produce IPP, which is catalyzed by mevalonate-5-diphosphate decarboxylase or diphosphomevalonate decarboxylase (PMD), which could be encoded by MVD1. IPP and DMAPP may establish a chemical equilibrium and interconvert with one another, which is catalyzed by idi, consistent with details described above. The idi enzyme could be encoded by IDI1. DMAPP may be converted to isoprene, which is catalyzed by IspS, consistent with details described above. In some cases, the MVA pathway may be further categorized into the “upper pathway”, which converts A-CoA to MVA, and the “lower pathway”, which produces DMAPP from MVA. In some cases, the MVA pathway may be introduced (i.e., cloned) into bacteria such as E. coli (which only has a native MEP pathway) as two synthetic operons, using a combination of bacterial and yeast enzymes. As a nonlimiting example, the lower pathway operon may be integrated into the chromosome while the upper pathway operon may be expressed on one or more plasmids, optionally alongside an IspS-encoding gene or the like. Chemical structures of each reactant, intermediate, and product of the MVP pathway are illustrated in Scheme 2 below:

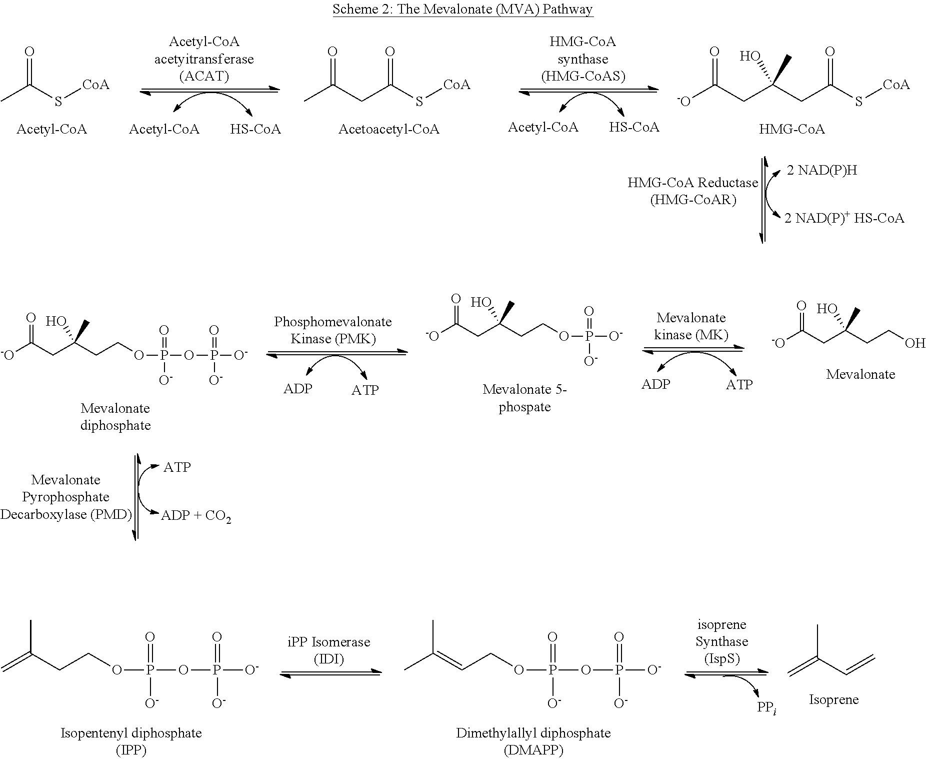

With continued reference to FIG. 2, scheme 200 includes a plurality of competing pathways that may limit the yield of isoprene production. IPP and/or DMAPP may be converted to geraniol and/or farnesol, which is catalyzed by enzymes such as without limitation geranyl diphosphate synthase (IspA), geranyl diphosphate synthase (GPPS), and/or farnesyl diphosphate synthase (FPPS). IPP and/or DMAPP may be converted to isoprenol, which is catalyzed by enzymes such as phosphatases and/or hydrolases including the Nudix hydrolase encoded by the NudB gene. As part of the central metabolic pathway, A-CoA may be converted to citrate to enter the tricarboxylic acid cycle, which is catalyzed by enzymes such as ATP citrate lyase (CitE) including AclY, AclB, and/or AclA. These pathways compete with the formation of isoprene. In some cases, to improve the yield of isoprene, byproduct isoprenol may be converted back to isoprene, which may be catalyzed by enzymes such as 3-methyl-3-buten-1-ol dehydroxylase (OhyA) or the like.

With continued reference to FIG. 2, isoprene is derived from intermediates in central metabolism. As described above, the first enzymes in both the MEP and MVA pathways use intermediates in central metabolism as substrates, namely, glyceraldehyde-3-phosphate+pyruvate or acetyl-CoA, respectively. These pathways have differing requirements for reducing power and energy. Thus, in addition to optimizing carbon flux through the MEP and/or MVA pathways, the levels of the central metabolism intermediates may also be maximized, and the reducing power/energy balance may be optimized by metabolic engineering. Additional details will be provided below in this disclosure.

With continued reference to FIG. 2, in one or more embodiments, to create a genetically engineered peptide superstructure for production of isoprene, a gene that encodes an idi may be isolated from E. coli. The resulting idi may accordingly be termed Ec-idi (SEQ ID NO: 30). Alternatively, in one or more embodiments, to create a genetically engineered peptide superstructure for production of isoprene, a gene that encodes an idi may be isolated from S. cerevisiae. The resulting idi may accordingly be termed Sc_idi (SEQ ID NO: 31). Similarly, in one or more embodiments, to create a genetically engineered peptide superstructure for production of isoprene, a gene that encodes an IspS may be sourced from Populus alba (P. alba). The resulting IspS may accordingly be termed Pa-IspS (SEQ ID NO: 25). Alternatively, in one or more embodiments, to create a genetically engineered peptide superstructure for production of isoprene, a gene that encodes an IspS may be sourced from Pueraria montana (P. montana). Specifically, in some cases, an IspS-encoding gene from P. montana may be further truncated to improve the solubility and/or activity of the IspS it encodes. As a nonlimiting example, this gene may be truncated at its 5′ end to produce a truncated IspS. The resulting truncated IspS may accordingly be termed tPm-IspS (SEQ ID NO: 26). In some cases, the sequence that encodes an idi may be located either upstream or downstream with respect to the sequence that encodes an IspS, with a sequence encoding a peptide linker (SEQ ID NO: 27-29) in between, consistent with details described above. As a nonlimiting example, a permutation between 2 types of idi (SEQ ID NO: 30-31), 2 types of IspS (SEQ ID NO: 25-26), 3 types of peptide linkers (SEQ ID NO: 27-29), and 2 types of arrangements (IspS-linker-idi or idi-linker-IspS) may create a library of 24 candidate gene sequences that encode a peptide superstructure (SEQ ID NO: 1-24). It is worth noting that the designation of first portion, second portion, first binding site, and second binding site, among others, is arbitrary. As a nonlimiting example, a nucleic acid sequence encoding first portion of peptide superstructure may be located either upstream or downstream, from the 5′ end to the 3′ end of heterologous nucleic acid construct, with respect to a nucleic acid sequence encoding second portion of the peptide superstructure.

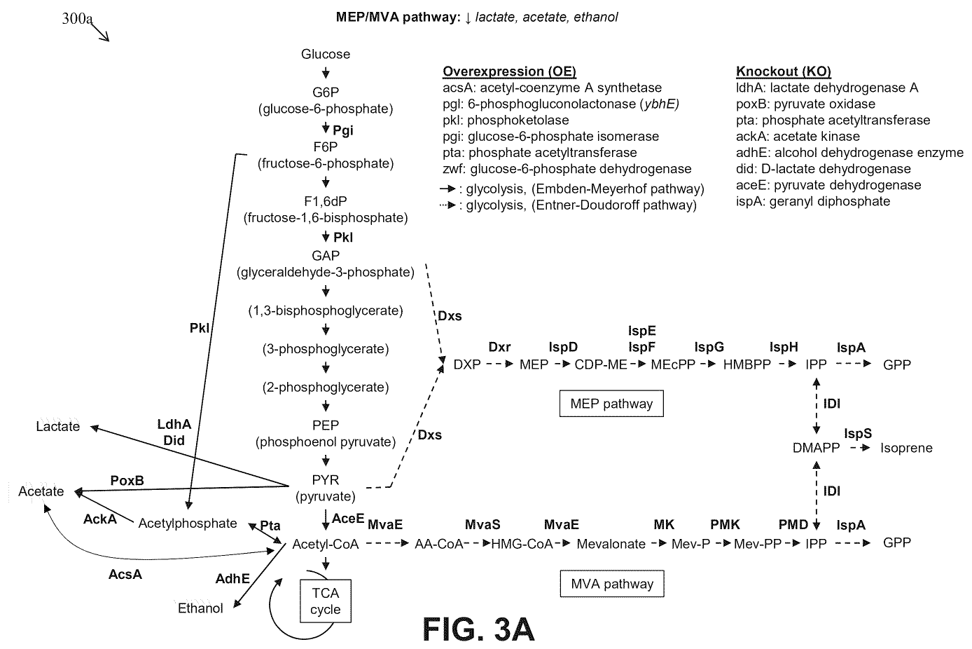

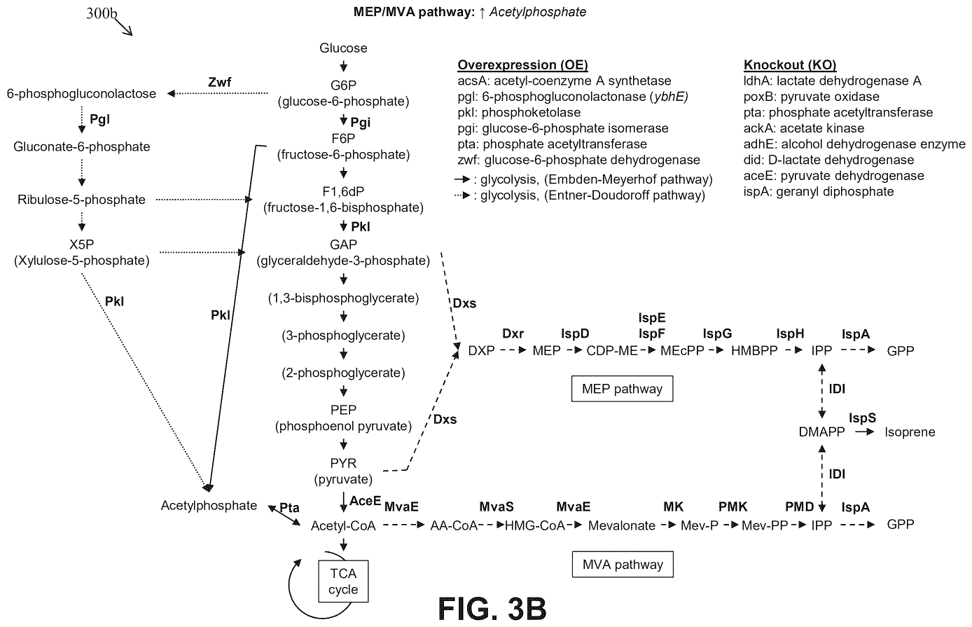

Referring now to FIGS. 3A-B, exemplary embodiments 300a-b of expanded metabolic pathways pertaining to isoprene biosynthesis are illustrated. FIGS. 3A-B include a plurality of potential gene targets. These gene targets and the activity thereof may be upregulated (for example, overexpressed), downregulated (for example, knocked down (KD)/knocked out (KO)), or otherwise modified, either individually or in combination, to increase the yield of isoprene.

With continued reference to FIGS. 3A-B, in one or more embodiments, the inherent activity of an enzyme may be improved by modifying the nucleic acid sequence that encodes the enzyme. Various techniques may be used to improve the inherent activity of an enzyme. As a nonlimiting example, directed evolution may be used to mimic natural selection in the lab by creating a large library of enzyme variants and selecting those with improved traits. As another nonlimiting example, site-directed mutagenesis may be used to introduce one or more specific mutations to enhance enzyme activity or stability. As another nonlimiting example, gene shuffling may be used to recombine segments of related genes to create new variants with enhanced properties. As another nonlimiting example, fusion proteins may be created by combining enzymes with other proteins to improve their function and/or stability. As another nonlimiting example, an enzyme such as DXS may be modified to lift one or more feedback regulations/inhibitions caused by an accumulation of its downstream products such as DMAPP, thereby improving the efficiency of isoprene synthesis. As a nonlimiting example, an enzyme such as OhyA or the like may be engineered to increase its inherent activity, thereby facilitating accumulation of isoprene while lifting the potentially toxic impact of isoprenol. As another nonlimiting example, an enzyme such MvaE or HMGR may be truncated from its naturally existing counterpart to improve its catalytic activity. As another nonlimiting example, one or more cofactors for one or more enzymes may be switched in order to improve the catalytic activity of an enzyme or enable fermentation conditions to be utilized that are more cost effective (e.g., with a lower oxygen requirement).

With continued reference to FIGS. 3A-B, in one or more embodiments, genes encoding enzymes such as 1-deoxy-D-xylulose-5-phosphate synthase (DXS), isoprene synthase (IspS), 3-hydroxy-3-methylglutaryl-coenzyme A reductase (HMG-CoA reductase or HMGR), 3-methyl-3-buten-1-ol dehydroxylase (OhyA) or OhyA-like enzymes, (E)-4-hydroxy-3-methylbut-2-enyl diphosphate synthase (IspG), 1-deoxy-D-xylulose 5-phosphate reductase (DXR), (E)-4-hydroxy-3-methylbut-2-enyl diphosphate reductase (IspH or HMBPP reductase), acetyl-CoA C-acetyltransferase/acetoacetyl-coenzyme A thiolase (MvaE, encoded by gene ERG10), HMG-CoA Synthase (MvaS, encoded by gene ERG13), acylating acetaldehyde dehydrogenase (A-ALD), among others, may be modified to increase the inherent activity of the enzyme, consistent with details described in the rest of this disclosure.

With continued reference to FIGS. 3A-B, in one or more embodiments, the activity of an enzyme may be modulated by increasing or upregulating an expression of a nucleic acid construct that encodes the enzyme. In some cases, a coding sequence may be configured to upregulate one or more genes encoding enzymes such as 3-methyl-3-buten-1-ol dehydroxylase (OhyA), transcription factor (encoded by UPC2), acetyl-CoA C-acetyltransferase/acetoacetyl-coenzyme A thiolase (MvaE, encoded by gene ERG10), mevalonate kinase (MK, encoded by ERG12), HMG-CoA synthase (MvaS, encoded by gene ERG13), aldehyde dehydrogenase (encoded by ALD5), acetyl-CoA synthetase (encoded by ACSA, ACS1, and/or ACS2, depending on the exact chassis used), pantothenate kinase (encoded by CAB1), dihydrolipoamide acetyltransferase component of the pyruvate dehydrogenase complex (encoded by LAT1), glucose-6-phosphate dehydrogenase (encoded by zwf), 6-phosphogluconolactonase (ybhE, encoded by pgl), phosphoketolase (encoded by pkl), glucose-6-phosphate isomerase (encoded by pgi), and phosphate acetyltransferase (encoded by pta), among others, as these genes encode enzymes that may contribute to the production and/or accumulation of isoprene. In some cases, regulation of a single gene may result in a regulation of multiple enzymes. As a nonlimiting example, when UPC2 is upregulated, ERG12 and ERG13 may also be overexpressed.

With continued reference to FIG. 3A-B, in one or more embodiments, the activity of an enzyme may be modulated by decreasing, attenuating, downregulating, or deleting the expression of a nucleic acid construct that encodes the enzyme. In some cases, a gene encoding an enzyme may be knocked down or knocked out. As nonlimiting examples, one or more genes encoding enzymes such as geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), pyruvate dehydrogenase (encoded by PDH) including pyruvate dehydrogenase E1 component (encoded by AceE), pyruvate oxidase (encoded by poxB), pyruvate decarboxylase (encoded by PDC), malate synthase (encoded by AceB or MLS1), citrate synthase (encoded by CIT1), ATP-citrate lyase (CitE) including AclY, AclB, and AcIA, glycerol-producing enzymes including cGPDH [40] and G3PP [41], ethanol-producing enzymes including invertase and zymase, alcohol dehydrogenase (encoded by ADH1), alcohol dehydrogenase enzyme (encoded by adhE), lactate-producing enzymes including lactate dehydrogenase such as lactate dehydrogenase A (encoded by IdhA) and D-lactate dehydrogenase (encoded by did), acetate-producing enzymes including acetyl coenzyme A (acetyl-CoA) hydrolases, acetate kinase (encoded by ackA), phosphatase, and/or hydrolase (for example, a hydrolase encoded by the NudB gene), among others, may be downregulated. However, it is worth noting that, in some cases, an enzyme may perform an essential function of a chassis and should not be completely knocked out or suppressed. As a nonlimiting example, FPPS encoded by ERG20 is essential to S. cerevisiae, and accordingly, specific sites within FPPS may be mutated in order to downregulate (not eliminate) its function. As another nonlimiting example, a diploid strain may be used to knockout one copy of ERG20 gene in order to downregulate the activity of the enzyme it encodes. In some cases, an enzyme may not be essential to a chassis and may be knocked out without causing adverse effects. As a nonlimiting example, genes such as MLS1 and CIT1 may be knocked out in such manner.

With continued reference to FIG. 3A-B, in some cases, multiple upregulations and downregulations may be implemented simultaneously to achieve a synergic effect. As a nonlimiting example, gene targets such as zwf, pgl, and/or pkl may be upregulated simultaneously to utilize the Entner-Doudoroff pathway in addition to the Embden-Meyerhof pathway regulated by pgi and pkl, thereby increasing the carbon flux of biosynthesis and increasing the yield of acetylphosphate (see FIG. 3A, embodiment 300a). As another nonlimiting example, gene targets such as ackA-pta, poxB, and/or ldhA may be knocked down or knocked out simultaneously to suppress competing reactions that form acetate, lactate, and/or ethanol (see FIG. 3B, embodiment 300b). As another nonlimiting example, simultaneous upregulation of ALD5 and downregulation of ADH1 may be combined to prevent acetyl-CoA from converting to ethanol via fermentation. Table 1 summarizes exemplary gene targets for upregulation and downregulation for S. cerevisiae and E. coli.

TABLE 1
Gene Targets for Improving Isoprene Production in S. cerevisiae and E. coli.

| | | Type of Genetic | |
|---|---|---|---|
| | | Modification | |
| | | Note: | |
| | | Knockout (KO) | |
| | | Knockdown (KD) | |
| Organism | Gene | Overexpress (OE) | Rationale |
| S. cerevisiae | ERG20: farnesyl | KD or mutate specific | Essential Gene; increase |
| | pyrophosphate synthetase | sites | DMAPP |
| S. cerevisiae | BTS1: geranylgeranyl | KO | Increase DMAPP |
| | diphosphate synthase | | |
| S. cerevisiae | UPC2: transcription factor | OE | Increase products of |
| | | | MVA pathway |
| S. cerevisiae | ERG10: acetyl-CoA C- | OE | Increase products of |
| | acetyltransferase | | MVA pathway |
| S. cerevisiae | ERG12: Mevalonate kinase | OE | Increase products of |
| S. cerevisiae | ERG13: HMG-CoA synthase | OE | MVA pathway |
| S. cerevisiae | ALD5: aldehyde | OE | Increase flux from |
| | dehydrogenases | | acetaldehyde to acetyl- |
| | | | CoA instead of ethanol |
| | | | production. Could also |
| | | | work with fed ethanol to |
| | | | push towards acety-CoA |
| S. cerevisiae | ADH1: alcohol dehydrogenase | KO | Increase flux from |
| | | | acetaldehyde to acetyl- |
| | | | CoA instead of ethanol |
| | | | production. Could also |
| | | | work with fed ethanol to |
| | | | push towards acety-CoA |
| S. cerevisiae | ACS1 and ACS2: acetyl-CoA | OE | Increase flux from |
| | synthetase | | acetaldehyde to acetyl- |
| | | | CoA instead of ethanol |
| | | | production. Could also |
| | | | work wih fed ethanol to |
| | | | push towards acety-CoA |
| S. cerevisiae | MLS1: Malate synthase | KO | Increase acetyl CoA pool |
| S. cerevisiae | CIT1: Citrate synthase | KO | Increase acetyl CoA pool |
| S. cerevisiae | CAB1: Pantothenate kinase | OE | Increase acetyl CoA pool |
| S. cerevisiae | LAT1: dihydrolipoamide | OE | Increase acetyl CoA pool |
| | acetyltransferase component | | |
| | of the pyruvate dehydrogenase | | |
| | complex | | |
| E. coli | zwf: glucose-6-phosphate | OE | Increase carbon flux to |
| | dehydrogenase | | acetylphosphate |
| | | | (overexpression of the |
| | | | Entner-Doudoroff |
| | | | pathway) |
| E. coli | acsA: acetyl-coenzyme A | OE | Increase carbon flux |
| | synthetase | | from acetate to acetyl- |
| | | | CoA |
| E. coli | pgl: 6- | OE | Increase carbon flux to |
| | phosphogluconolactonase | | acetylphosphate |
| | (ybhE) | | (overexpression of the |
| | | | Entner-Doudoroff |
| | | | pathway) |
| E. coli | pkl: phosphoketolase | OE | Increase carbon flux to |
| | | | acetylphosphate |
| | | | (overexpression of the |
| | | | Entner-Doudoroff |
| | | | pathway) |
| E. coli | pgi: glucose-6-phosphate | OE | Increase carbon flux |
| | isomerase | | from G6P to F6P |
| E. coli | pta: phosphate | OE | Increase acetyl-CoA |
| | acetyltransferase | | from acetylphosphate |
| E. coli | ldhA: lactate dehydrogenase A | KO/KD | Reduce lactate |
| | | | production from |
| | | | pyruvate |
| E. coli | poxB: pyruvate oxidase | KO/KD | Reduce acetate |
| | | | production from |
| | | | pyruvate |
| E. coli | ackA: acetate kinase | KO/KD | Reduce acetate |
| | | | production from |
| | | | acetylphosphate |
| E. coli | adhE: alcohol dehydrogenase | KO/KD | Reduce ethanol |
| | enzyme | | production from acetyl- |
| | | | CoA |
| E. coli | did: D-lactate dehydrogenase | KO/KD | Reduce lactate |
| | | | production from |
| | | | pyruvate |
| E. coli | aceE: pyruvate dehydrogenase | KD | Increase carbon flux to |
| | | | MEP pathway from |
| | | | pyruvate |
| E. coli | ispA: geranyl diphosphate | KD | Increase carbon flux to |
| | | | DMAPP from IPP and |
| | | | reduce it to GPP from |
| | | | IPP |

With continued reference to FIGS. 3A-B, in one or more embodiments, promoter engineering may be used to improve the transcriptional level of a gene. Promoter engineering modifies the promoter region to increase gene expression levels, thereby improving enzyme production and activity. In one or more embodiments, codon optimization may be used to improve the translational efficiency of a gene. For the purposes of this disclosure, “codon optimization” is a technique used in genetic engineering to improve the expression of a gene in a particular host organism. Codon optimization involves altering the DNA sequence of a gene to use codons that are more frequently preferred by the host organism's translational machinery. A codon optimization process may take into account a codon bias of the host, ensuring that a synthetic gene sequence is translated more efficiently into the desired protein. Codon optimization may enhance the yield and function of a protein, which may be crucial for various applications in biotechnology and synthetic biology. It is worth noting that promoter engineering and codon optimization are distinct yet complementary techniques in genetic engineering. Promoter engineering involves modifying the promoter region of a gene to enhance its expression by improving the binding efficiency of transcriptional machinery. Codon optimization, on the other hand, focuses on altering the coding sequence of a gene to use preferred codons of the host organism, thereby improving translation efficiency. While both aim to increase protein production, promoter engineering targets transcriptional levels, and codon optimization targets translational efficiency. Combining both techniques may in some cases synergistically enhance an overall gene expression.

With continued reference to FIGS. 3A-B, in some cases, a gene encoding an enzyme may be silenced using Hfq-mediated RNA silencing. For the purposes of this disclosure, “Hfq-mediated RNA silencing” is a process wherein the Hfq protein facilitates a regulation of gene expression through RNA interactions, by binding to small regulatory RNAs (sRNAs) and messenger RNAs (mRNAs) to promote a formation of RNA duplexes. This interaction may enhance or inhibit a translation of target mRNAs, leading to gene silencing. In some cases, this process may be useful for post-transcriptional regulation and may be harnessed for genetic engineering, therapeutic interventions, and synthetic biology applications. As nonlimiting examples, to facilitate accumulation of isoprene, Hfq-mediated RNA silencing may be used against geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), malate synthase (encoded by AceB or MLS1), ATP-citrate lyase (CitE) including AclY, AclB, and AcIA, and/or pyruvate dehydrogenase (encoded by PDH) including pyruvate dehydrogenase E1 component (encoded by AceE), among others, to downregulate its activity, consistent with details described above.

With continued reference to FIGS. 3A-B, in some cases, one or more pathways within schemes of embodiments 300a or 300b may be condensed, inhibited, rearranged, or otherwise modified. As a nonlimiting example, one or more genes encoding one or more enzymes may be modified to provide a phosphoketolase workaround, such as by upregulating glycolysis, the pentose phosphate pathway, or the Entner-Doudoroff pathway. As another nonlimiting example, one or more enzymes may be co-localized to create multiple reaction centers in tandem, thereby bypassing one or more competing side reactions, consistent with details described above. As another nonlimiting example, one or more genes encoding one or more enzymes may be modified to inhibit the phosphotransacetylase-acetate kinase (Pta-AckA) pathway to facilitate acetate reuptake in a Crabtree-positive enzyme. The Pta-AckA pathway is a key metabolic route in many bacteria and may be particularly significant under anaerobic conditions or when there is an excess of carbon sources, as it allows a cell to dispose of excess A-CoA and generate additional ATP. Inhibition of the Pta-AckA pathway may facilitate an acetate reuptake and improve the overall carbon efficiency of isoprene production. The Pta-AckA pathway involves two main enzymes: Pta catalyzes the conversion of A-CoA to acetyl phosphate, whereas AckA catalyzes the conversion of acetyl phosphate to acetate to generate ATP. By inhibiting these enzymes, the production of acetate may be reduced. Such reduction may be beneficial in preventing acetate accumulation that can be detrimental to cellular growth and productivity. Genetically engineered microbe may be configured to produce a Pta-AckA inhibitor. For the purposes of this disclosure, a “phosphotransacetylase-acetate kinase inhibitor” is a chemical compound that interferes with the activity of the phosphotransacetylase (Pta) and/or acetate kinase (AckA). Nonlimiting examples of Pta-AckA inhibitors may include deacetylase inhibitors such as tricostatin A and nicotinamide. Specifically, such inhibitors may function by regulating acetyl phosphate-dependent protein acetylation. As another nonlimiting example, the MEP and the MVA pathways may be balanced with each other. Specifically, leveraging the MEP and MVA pathways may enhance the yield and production efficiency of isoprene by optimizing the flux of metabolites, providing metabolic redundancy, and improving the robustness of the production strain under varying conditions. In some cases, such balanced pathways may be implemented using a shunt. Such balanced pathways may offer several benefits. As a nonlimiting example, such balanced pathways may lead to a more efficient utilization of substrates, thereby reducing waste and improving the carbon economy of a genetically engineered microbe. Additionally, as a further nonlimiting example, such balanced pathways may allow for finer control over metabolic fluxes through genetic and regulatory modifications, facilitating the tuning of pathway activities to match specific production needs. Overall, such balanced pathways may enhance the efficiency, robustness, and flexibility of isoprene production, making it more economically viable and sustainable for industrial applications. As another nonlimiting example, the MEP and the MVA pathways may be balanced by increasing a flux through the MEP pathway to reduce carbon loss to the TCA cycle; this strategy may lead to an increase in isoprene production. A theoretical calculation of isoprene yield based upon flux ratios through the MEP and MVA pathways suggests that isoprene yield may increase with an increasing flux along the MEP pathway, with a maximum yield of approximately 0.31 g of isoprene per gram of glucose when the flux ratio is approximately 60:40.

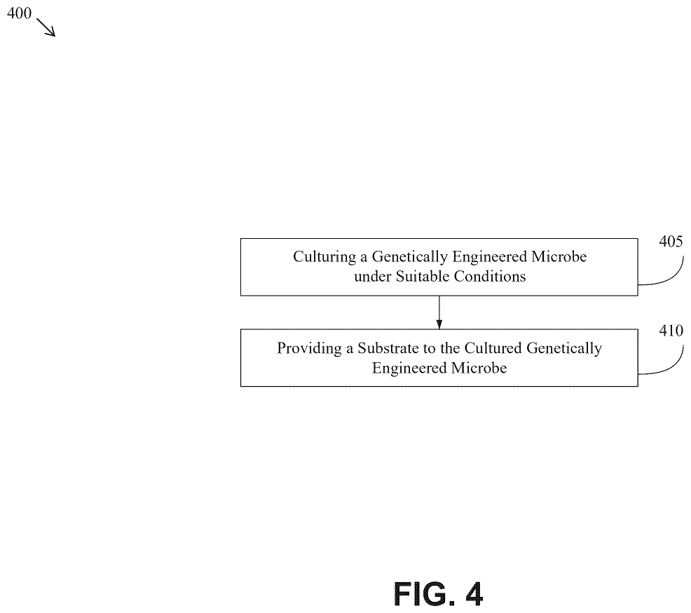

Referring now to FIG. 4, a method 400 for producing isoprene using genetically engineered peptide superstructure is illustrated. At step 405, method 400 includes culturing a genetically engineered microbe under suitable conditions, consistent with details described above without limitation. For the purposes of this disclosure, a “suitable condition” is an environmental condition or factor suitable for the growth and/or replication of a microbe. In some cases, a suitable condition may vary from one type of microbe to another. A suitable condition may include without limitation a suitable temperature/temperature range, a suitable pressure/pressure range, a suitable pH or pH range, a suitable ionic strength/range of ionic strength, a suitable osmotic pressure/range of osmotic pressure, a suitable concentration/concentration range of one or more nutrients, a suitable level of metabolic waste/metabolites, among others. A person of ordinary skill in the art, upon reviewing the entirety of this disclosure, will be able to identify suitable conditions specific to one or more microbes described herein.

With continued reference to FIG. 4, at step 410, method further includes providing substrate to genetically engineered microbe to produce isoprene, consistent with details described above without limitation. Specifically, first binding site is configured to bind to substrate and catalyze first chemical reaction that converts the substrate to intermediate. Similarly, second binding site is configured to catalyze second chemical reaction that converts intermediate to isoprene. In some cases, produced isoprene may be isolated and/or purified using an absorbing/stripping apparatus, consistent with details disclosed in U.S. patent application Ser. No. 18/928,691 (attorney docket number 1656-001USU1), filed on Oct. 28, 2024, entitled “SYSTEMS AND METHODS FOR PRODUCING A DIMETHYLCYCLOOCTANE-BASED AVIATION FUEL FROM ISOPRENE”, the entirety of which is incorporated herein by reference.

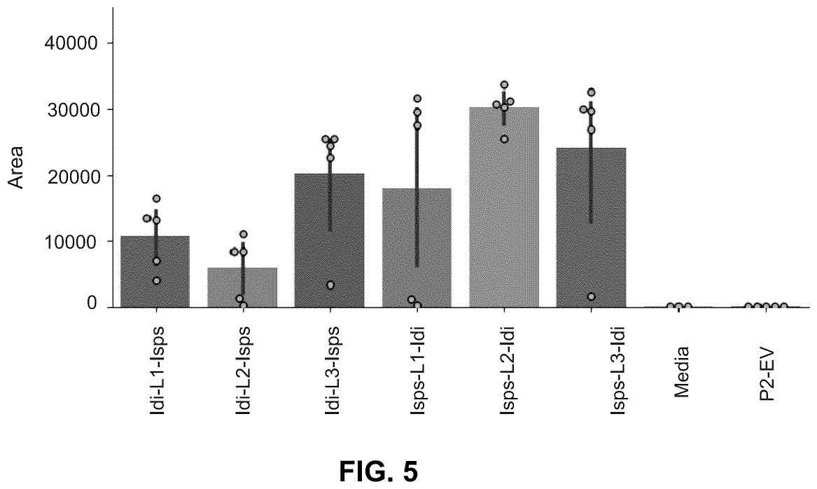

With continued reference to FIG. 5, exemplary experimental result of isoprene production for IDI and IspS fusion, media only, empty vector (P1+P2 system without IspS) control is illustrated. The x-axis represents six IDI and IspS fusion construct design along with media only control, and empty vector control (P1+P2 system without IspS). The y-axis represents isoprene production detected by area using gas chromatography. All fusion constructs produced isoprene. Each IspS_IDI construct produced more isoprene than its reversed counterpart, IDI_IspS. In the IDI_IspS orientation, the linker 2 construct (flexible linker) produced most isoprene. In the IspS_IDI orientation, the linker 3 construct (rigid linker) produced the most isoprene.

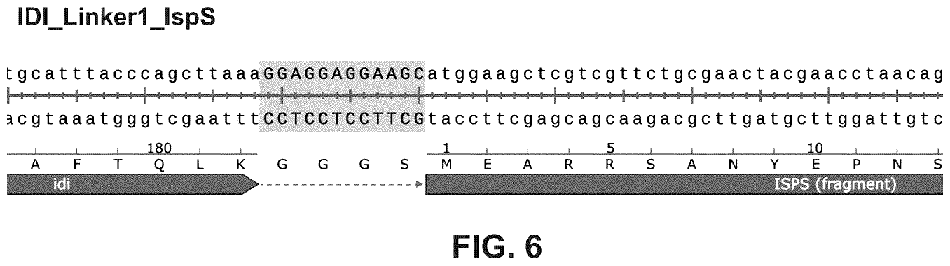

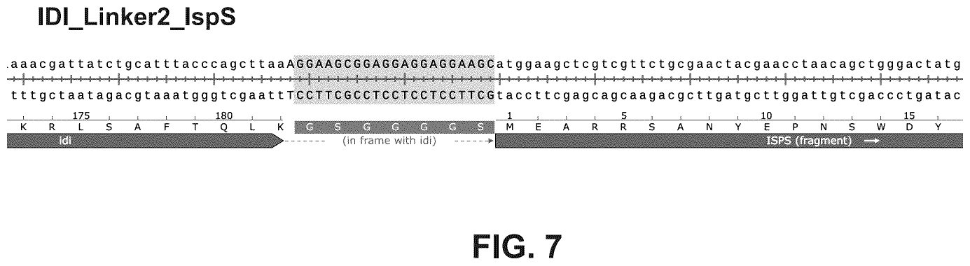

With continued reference to FIG. 6, exemplary result of the fusion construct containing the IDI_Linker1_IspS sequence is illustrated. Linker 1 (Ec-Idi Linker-1 Pa-IspS, SEQ: 1) has an amino acid sequence of “GGGS,” wherein G is glycine and S is serine. In the construct, the IDI sequence begins at the N-terminal, and the IspS sequence terminates at the C-terminal. Linker 1 provides flexibility between the two enzyme domains.

With continued reference to FIG. 7, exemplary result of the fusion construct containing the IDI_Linker2_IspS sequence is illustrated. Linker 2 (Ec-Idi Linker-2 Pa-IspS, SEQ: 3) has an amino acid sequence of “GSGGGGS,” wherein G is glycine and S is serine. In the construct, the IDI sequence begins at the N-terminal, and the IspS sequence terminates at the C-terminal. Linker 2 provides flexibility between the two enzyme domains.

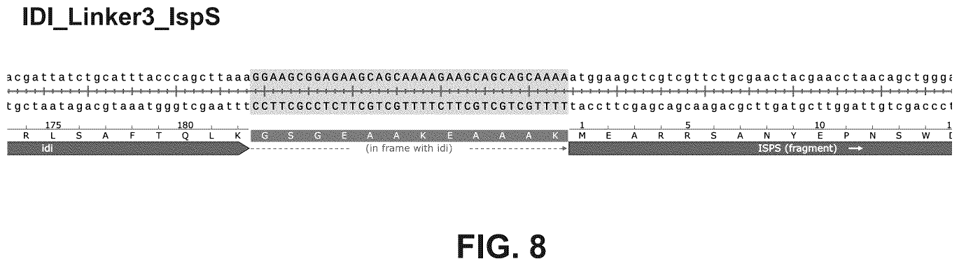

With continued reference to FIG. 8, exemplary result of the fusion construct containing the IDI_Linker3_IspS sequence is illustrated. Linker 3 (Ec-Idi Linker-3 Pa-IspS, SEQ: 5) has an amino acid sequence of “GSGEAAKEAAAK,” wherein G is glycine, S is serine, E is glutamic acid, A is alanine, and K is lysine. In the construct, the IDI sequence begins at the N-terminal, and the IspS sequence terminates at the C-terminal. Linker 3 imparts rigidity between the two enzyme domains, as the residues glutamic acid, alanine, and lysine promote alpha-helical geometry in the linker region, thereby limiting domain flexibility.

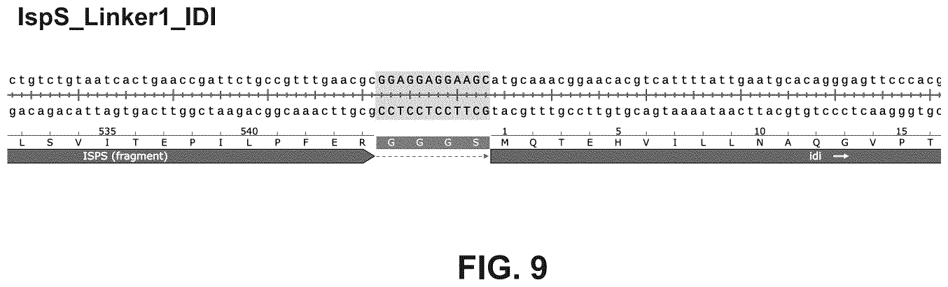

With continued reference to FIG. 9, exemplary result of the fusion construct containing the IspS_Linker1_IDI sequence is illustrated. Linker 1 (Pa-IspS Linker-1 Ec-Idi, SEQ: 13) has an amino acid sequence of “GGGS,” wherein G is glycine and S is serine. In the construct, the IspS sequence begins at the N-terminal, and the IDI sequence terminates at the C-terminal. Linker 1 provides flexibility between the two enzyme domains.

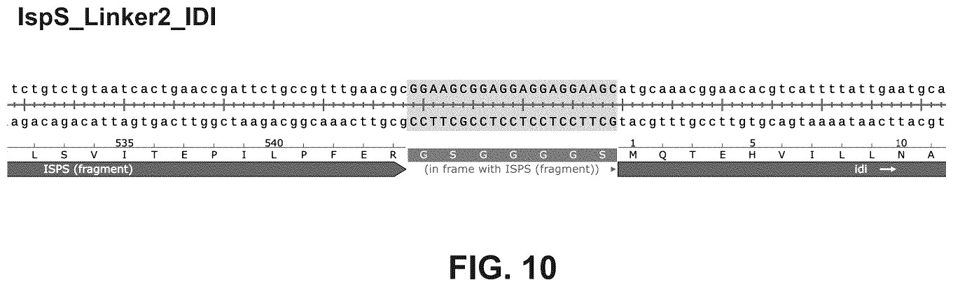

With continued reference to FIG. 10, exemplary result of the fusion construct containing the IspS_Linker2_IDI sequence is illustrated. Linker 2 (Pa-IspS Linker-2 Ec-Idi, SEQ: 15) has an amino acid sequence of “GSGGGGS,” wherein G is glycine and S is serine. In the construct, the IspS sequence begins at the N-terminal, and the IDI sequence terminates at the C-terminal. Linker 2 provides flexibility between the two enzyme domains.

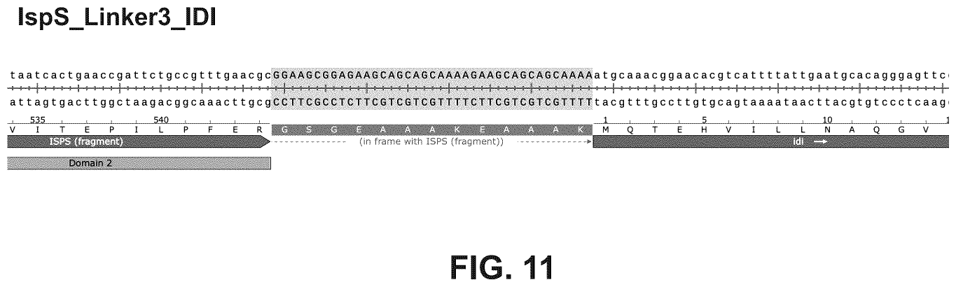

With continued reference to FIG. 11, exemplary result of the fusion construct containing the IspS_Linker3_IDI sequence is illustrated. Linker 3 (Pa-IspS Linker-3 Ec-Idi, SEQ: 17) has an amino acid sequence of “GSGEAAKEAAAK,” wherein G is glycine, S is serine, E is glutamic acid, A is alanine, and K is lysine. In the construct, the IspS sequence begins at the N-terminal, and the IDI sequence terminates at the C-terminal. Linker 3 imparts rigidity between the two enzyme domains, as the residues glutamic acid, alanine, and lysine promote alpha-helical geometry in the linker region, thereby limiting domain flexibility.

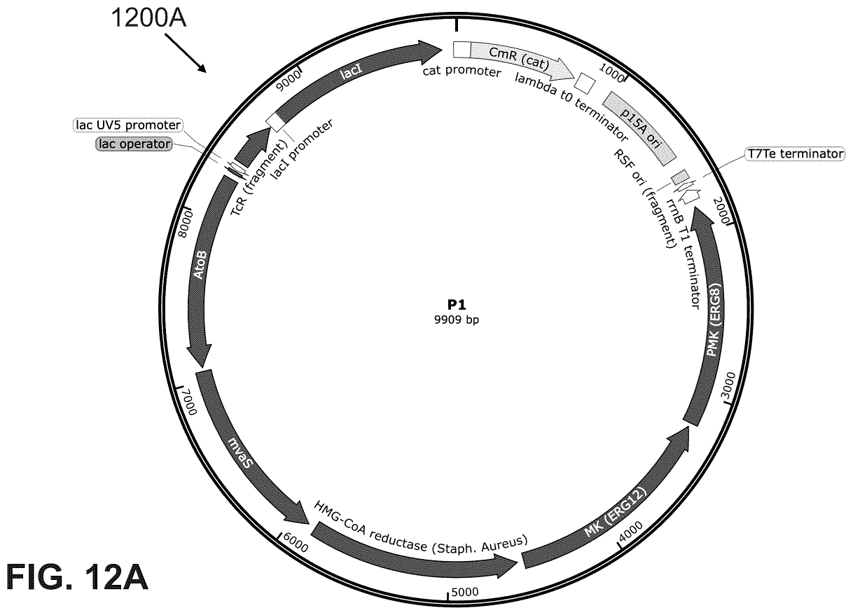

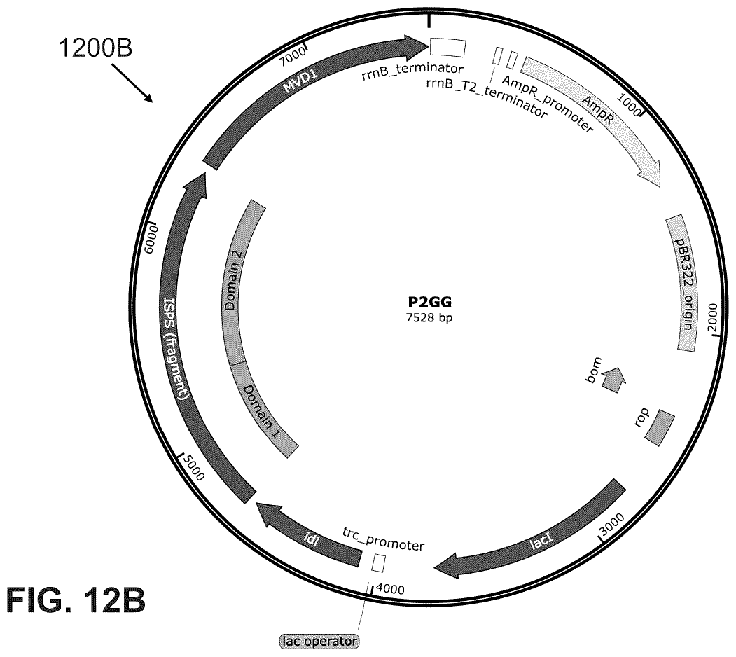

With continued reference to FIGS. 12A-B, exemplary embodiments of plasmid vector maps used for P1+P2 systems are illustrated. P1+P2 system includes two plasmids: P1 system (1200A) (P1 System, SEQ: 32) and P2 system (1200B) (P2 system, SEQ: 33) that contain genes to encode all the enzymes in the MVA pathway. P1 plasmid (1200B) encode enzymes to be heterologously expressed in upper mevalonate pathway in E. coli. The genes AtoB, MvaS, HMGR, MK (ERG12), and PMK (ERG8) are encoded on the plasmid P1 (1200A), and translate the proteins acetyl-coA acetyltransferase, HMG-CoA synthase, HMG-CoA reductase, mevalonate kinase, and phosphomevalonate kinase respectively. The genes encoded by P1 are under control of the lacUV5 promoter. The P1 plasmid (1200A) as a p15A origin of replication and a chloramphenicol resistance marker. The P2 plasmid (1200B) includes of the genes idi, IspS and MVD1, when encode isopentenyl diphosphate isomerase, isoprene synthase and mevalonate decarboxylase respectively. P2 plasmid (1200B) and P2GG plasmid (P2GG system, SEQ: 34) encod the enzymes to be heterologoulsly expressed in the lower mevalonate pathway in E. coli. The P2 plasmid (1200B) was modified to simplify downstream cloning by removal of BsaI restriction sites to yield the P2GG plasmid. The P2GG plasmid encodes the same genes as P2, which in both cases are under control of the Trc promoter. Both P2 and P2GG have a pBR332 origin of replication and an ampicillin resistance marker.

### EXAMPLES

### Example 1: Identify Chassis for Production of Isoprene and Improve Productivity and Yield of Isoprene in E. coli

### Purpose of the Study

This study investigates the amount of isoprene produced by the IspS_IDI fusion construct using recombinant DNA techniques, and examines how different linkers and orientations of the fusion construct impact isoprene production.

### Introduction

Isopentyl diphosphate isomerase (IDI) and Isoprene synthase (IspS) are the two final enzymes in the mevalonate (MVA) pathway for isoprene synthesis and in the pathway contained in the P1+P2 plasmid system used by CLEANJOULE to investigate pathway improvement at this time. The P1+P2 system includes two plasmids (P1 and P2 respectively) that contain genes to encode all the enzymes in the MVA pathway. The genes AtoB, MvaS, HMGR, MK (ERG12), and PMK (ERG8) are encoded on the plasmid P1, and translate the proteins acetyl-coA acetyltransferase, HMG-CoA synthase, HMG-CoA reductase, mevalonate kinase, and phosphomevalonate kinase respectively. The genes encoded by P1 are under control of the lacUV5 promoter. The P1 plasmid as a p15A origin of replication and a chloramphenicol resistance marker. The P2 plasmid includes of the genes idi, IspS and MVD1, when encode isopentenyl diphosphate isomerase, isoprene synthase and mevalonate decarboxylase respectively. The P2 plasmid was modified to simplify downstream cloning by removal of BsaI restriction sites to yield the P2GG plasmid. The P2GG plasmid encodes the same genes as P2, which in both cases are under control of the Trc promoter. Both P2 and P2GG have a pBR332 origin of replication and an ampicillin resistance marker. We hypothesize that fusions of IspS and IDI will improve pathway flux at their respective steps because they are involved in sequential steps in the pathway, and that could allow the fusion protein to be more closely associate with its sequential substrates.

### Summary of Study

Six enzyme-linker constructs were designed and produced detectable levels of isoprene. Each IspS_IDI construct generated more isoprene than its reversed counterpart, IDI_IspS.

### Strategies

For our case, three linker sequences were chosen: the first two are short and long GS linkers (flexible) and the third is an EAAAK linker (rigid). The sequences for Linker 1, Linker 2, and Linker 3 are GGGS, GSGGGGS, and GSGEAAAKEAAAK, respectively.

The fusions of IDI and IspS are detailed in Table 2. Fusion proteins were created using the P2 plasmid as a template, where IDI and IspS nucleotide sequences were replaced by the fusion protein sequence and appropriate ribosome binding site (RBS) with the stop codon at the end of the first protein omitted while preserving the start codon at the start of the second protein in the sequence.

TABLE 2
Fusion Constructs & Construction Details

| | | | Sequencing | Glycerol |
|---|---|---|---|---|
| Fusion | Order (N to C) | SnapGene File | Verification# | Stock # |
| L0⁴ | IDI_Linker1_IspS | P2_Fusion.dna | 2272821 | s56.1 (P2 |
| | | | (Eurofins) | Fusion) |
| L1⁵ | IDI_Linker2_IspS | AZ821_L1_Assembly.dna | 52FZ9Q | A7.8 |
| | | | (Plasmidsaurus) | |
| L2⁵ | IDI_Linker3_IspS | AZ821-L2-Assembly.dna | 2FVJ3W | A8.16 |
| | | | (Plasmidsaurus) | |
| L3⁶ | IspS_Linker1_IDI | P2_IspS-Linker_1-Idi.dna | 373BFW | A9.1, |
| | | | (Plasmidsaurus) | A9.2 |
| L4⁶ | IspS_Linker2_IDI | P2_IspS-Linker_2-Idi.dna | 373BFW | A10.1, |
| | | | (Plasmidsaurus) | A10.2 |
| L5⁶ | IspS_Linker3_IDI | P2_IspS-Linker_3-Idi.dna | 373BFW | A11.2 |
| | | | (Plasmidsaurus) | |

All fusion constructs produced isoprene. Each IspS_IDI construct produced more isoprene than its reversed counterpart, IDI_IspS. In the IDI_IspS orientation, the Linker 2 construct (flexible) produced most isoprene. In the IspS_IDI orientation, the Linker 3 construct (rigid) produced the most isoprene.

### Conclusion

All IspS_IDI fusion constructs produced isoprene.

### Materials & Methods

### Cloning

P2GG was amplified at IDI using primers designed for domesticated golden gate assembly. Each pair of primers contains a BsaI restriction site, a complimentary overhang to the adjacent segment, the appropriate linker sequence, and a complimentary annealing region. L0 was previously constructed. L3-5 were assembled by amplifying the whole backbone from P2GG with primers that contained the linker sequences and performing a Golden-Gate mediated self-ligation. Self-ligation failed for the construction of L1 and L2, and primers were redesigned to construct L1 and L2 through golden-gate mediated 2-fragment assembly. The assembled products were transformed into DH5-alpha competent cells and colonies were screened by colony PCR. Successful colonies were sequence verified. Glycerol stocks are available for all constructs (see Table 2 for strain codes).

### Expression

Plasmids containing each of the fusion combinations mentioned above were transformed into BL21 (DE3) competent cells along with P1. Colonies were then selected and grown in liquid culture (2 mL LB+100 μg/mL Carbenicillin+35 μg/mL Chloramphenicol+10 g/L glucose) at 37° C. until the OD was approximately 0.6. At this point, 790 μL of the cells were transferred to autosampler vials and induced by addition of 7.9 μL of 100 mM IPTG. The tubes were then sealed and incubated at 30° C. for roughly 48 hours, at which point the headspace from the tubes was sampled and analyzed by GC-FID with the autosampler program H-Isoprene-1 Method. A single expression experiment was performed with 5 biological replicates for each sample. Expression data is in Table 3.

TABLE 3
Expression Details

| | Sample | | | | | | |
|---|---|---|---|---|---|---|---|
| Sample # | Label | RT | Width | Area | Height | Area % | Induction O.D. |
| 1 | P2-EV | 1.02 | 0.051 | 128 | 41 | 100 | 0.6 |
| 2 | P2-EV | 1.02 | 0.054 | 116 | 36 | 100 | 0.5 |
| 3 | P2-EV | 1.02 | 0.054 | 137 | 43 | 100 | 0.8 |
| 4 | P2-EV | 1.02 | 0.052 | 154 | 49 | 100 | 0.6 |
| 5 | P2-EV | 1.02 | 0.054 | 169 | 51 | 100 | 0.6 |
| 6 | Idi-L1-IspS | 1.03 | 0.051 | 16378 | 5381 | 100 | 0.9 |
| 7 | Idi-L1-IspS | 1.03 | 0.051 | 13218 | 4339 | 100 | 0.7 |
| 8 | Idi-L1-IspS | 1.03 | 0.051 | 13467 | 4425 | 100 | 0.7 |
| 9 | Idi-L1-IspS | 1.03 | 0.051 | 7013 | 2307 | 100 | 0.6 |
| 10 | Idi-L1-IspS | 1.03 | 0.05 | 3986 | 1316 | 100 | 0.7 |
| 11 | Idi-L2-IspS | 1.02 | 0.051 | 260 | 81 | 100 | 0.6 |
| 12 | Idi-L2-IspS | 1.03 | 0.051 | 1384 | 451 | 100 | 0.7 |
| 13 | Idi-L2-IspS | 1.03 | 0.051 | 8416 | 2759 | 100 | 0.6 |
| 14 | Idi-L2-IspS | 1.03 | 0.051 | 11066 | 3637 | 100 | 0.6 |
| 15 | Idi-L2-IspS | 1.03 | 0.051 | 8482 | 2785 | 100 | 0.5 |
| 16 | Idi-L3-IspS | 1.03 | 0.051 | 25576 | 8414 | 100 | 0.7 |
| 17 | Idi-L3-IspS | 1.03 | 0.051 | 3305 | 1080 | 100 | 0.6 |
| 18 | Idi-L3-IspS | 1.03 | 0.051 | 24381 | 8010 | 100 | 1 |
| 19 | Idi-L3-IspS | 1.03 | 0.051 | 22584 | 7432 | 100 | 0.6 |
| 20 | Idi-L3-IspS | 1.03 | 0.051 | 25467 | 8376 | 100 | 0.6 |
| 21 | IspS-L1-Idi | 1.02 | 0.052 | 308 | 97 | 100 | 0.6 |
| 22 | IspS-L1-Idi | 1.03 | 0.051 | 31702 | 10394 | 100 | 0.6 |
| 23 | IspS-L1-Idi | 1.03 | 0.051 | 27533 | 9032 | 100 | 0.6 |
| 24 | IspS-L1-Idi | 1.03 | 0.051 | 1164 | 378 | 100 | 0.5 |
| 25 | IspS-L1-Idi | 1.03 | 0.051 | 29508 | 9700 | 100 | 0.7 |
| 26 | IspS-L2-Idi | 1.03 | 0.051 | 25509 | 8354 | 100 | 0.6 |
| 27 | IspS-L2-Idi | 1.03 | 0.05 | 31208 | 10283 | 100 | 0.5 |
| 28 | IspS-L2-Idi | 1.03 | 0.051 | 30778 | 10127 | 100 | 0.5 |
| 29 | IspS-L2-Idi | 1.03 | 0.051 | 30309 | 9959 | 100 | 0.6 |
| 30 | IspS-L2-Idi | 1.03 | 0.051 | 33686 | 11061 | 100 | 0.5 |
| 31 | IspS-L3-Idi | 1.03 | 0.051 | 26869 | 8827 | 100 | 1 |
| 32 | IspS-L3-Idi | 1.03 | 0.051 | 29611 | 9719 | 100 | 0.9 |
| 33 | IspS-L3-Idi | 1.03 | 0.051 | 1746 | 569 | 100 | 0.8 |
| 34 | IspS-L3-Idi | 1.03 | 0.051 | 29958 | 9835 | 100 | 1.1 |
| 35 | IspS-L3-Idi | 1.03 | 0.051 | 32668 | 10741 | 100 | 0.9 |
| M1 | Media | 1.02 | 0.052 | 145 | 46 | 100 | 0.1 |
| M2 | Media | 1.02 | 0.052 | 139 | 44 | 100 | 0.2 |
| M3 | Media | 1.02 | 0.053 | 141 | 44 | 100 | 0.2 |
| AIR | Air | 0 | 0 | 0 | 0 | 0 | — |

### Fusion Construct Construction

All fusion constructs utilize the Trc promoter. The rrnB and rrnB_T2 terminators were used, terminating transcription after MVD1. The genes IDI, IspS, and MVD1 are arranged in a polycistronic configuration and are all transcribed under the control of the Trc promoter located upstream of IDI.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A genetically engineered microbe capable of producing isoprene, the genetically engineered microbe comprises:
a heterologous nucleic acid construct encoding at least a portion of a peptide superstructure, wherein the superstructure comprises:
a first portion comprising a first binding site configured to catalyze a first chemical reaction, wherein the first reaction converts a substrate to an intermediate; and
a second portion comprising a second binding site configured to catalyze a second chemical reaction, wherein:
the second binding site is located in proximity to the first binding site; and
the second chemical reaction converts the intermediate to isoprene.

2. The genetically engineered microbe of claim 1, wherein:
the first portion comprises at least a functional fragment of isopentenyl diphosphate (IPP) isomerase; and
the second portion comprises at least a functional fragment of isoprene synthase (IspS).

3. The genetically engineered microbe of claim 1, wherein:
the substrate comprises IPP; and
the intermediate comprises dimethylallyl pyrophosphate (DMAPP).

4. The genetically engineered microbe of claim 1, wherein the genetically engineered microbe comprises bacteria or fungi, wherein the bacteria or fungi includes one or more members selected from a group consisting of Escherichia Coli, Lactococcus, Vibrio natriegens, Saccharomyces cerevisiae, Aspergillus, Yarrowia lipolytica, Bacillus subtilis, Clostridium ljungdahlii, and Pseudomonas putid.

5. The genetically engineered microbe of claim 1, wherein the genetically engineered microbe comprises a Crabtree-negative microbe.

6. The genetically engineered microbe of claim 1, wherein the genetically engineered microbe comprises a diploid strain.

7. A genetically engineered peptide superstructure for production of isoprene, the superstructure comprising:
a first portion comprising at least a functional fragment of isoprene synthase (IspS) configured to catalyze a first chemical reaction; and
a second portion comprising at least a functional fragment of isopentenyl diphosphate (IPP) isomerase configured to catalyze a second chemical reaction, wherein the second chemical reaction converts a substrate to an intermediate, and wherein:
the second portion is covalently linked to the first portion through a linker; and
the intermediate is converted into isoprene by the first chemical reaction.

8. The superstructure of claim 7, wherein the first portion begins at N-terminal end of the superstructure and the second portion terminates at a C-terminal end of the superstructure.

9. The superstructure of claim 7, wherein:
the substrate comprises IPP; and
the intermediate comprises dimethylallyl pyrophosphate (DMAPP).

10. The superstructure of claim 7, wherein the linker comprises a rigid linker or a flexible linker.

11. The superstructure of claim 7, wherein a level of isoprene production is correlated to a rigidity of the linker.

12. A genetically engineered peptide superstructure for production of isoprene, the superstructure comprising:
a first portion comprising at least a functional fragment of isopentenyl diphosphate (IPP) isomerase configured to catalyze a first chemical reaction, wherein the first chemical reaction converts a substrate to an intermediate; and
a second portion comprising at least a functional fragment of isoprene synthase (IspS) configured to catalyze a second chemical reaction, wherein:
the second portion is covalently linked to the first portion through a linker; and
the intermediate is converted into isoprene by the second chemical reaction.

13. The superstructure of claim 12, wherein the first portion begins at N-terminal end of the superstructure and the second portion terminates at a C-terminal end of the superstructure.

14. The superstructure of claim 12, wherein:
the substrate comprises IPP; and
the intermediate comprises dimethylallyl pyrophosphate (DMAPP).

15. The superstructure of claim 12, wherein the linker comprises a rigid linker or a flexible linker.

16. A method of producing isoprene using a genetically engineered peptide superstructure, the method comprising:
culturing a genetically engineered microbe under suitable conditions, wherein:
the genetically engineered microbe comprises a heterologous nucleic acid construct encoding at least a portion of a peptide superstructure; and
the superstructure comprises:
a first portion comprising a first binding site; and
a second portion comprising a second binding site, the second portion is covalently linked to the first portion through a linker;
providing a substrate to the cultured genetically engineered microbe, wherein:
the first binding site is configured to bind to the substrate and catalyze a first chemical reaction that converts the substrate to an intermediate; and
the second binding site is configured to catalyze a second chemical reaction that converts the intermediate to isoprene.

17. The method of claim 16, wherein the genetically engineered microbe comprises Escherichia coli.

18. The method of claim 16, wherein:
the first portion comprises at least a functional fragment of isopentenyl diphosphate (IPP) isomerase; and
the second portion comprises at least a functional fragment of isoprene synthase (IspS).

19. The method of claim 16, wherein the first portion begins at C-terminal end of the superstructure and the second portion terminates at a N-terminal end of the superstructure.

20. The method of claim 16, wherein:
the substrate comprises IPP; and
the intermediate comprises dimethylallyl pyrophosphate (DMAPP).
